(19) United States
(12) Patent Application Publication
(10) Pub. No.: US 2026/0224494 A1
(43) Pub. Date: Aug. 6, 2026

(54) MILVEXIAN PHARMACEUTICAL COMPOSITIONS

(71) Applicants: BRISTOL-MYERS SQUIBB COMPANY, Princeton, NJ (US); JANSSEN PHARMACEUTICA NV, Beerse (BE)

(72) Inventors: Sune ANDERSEN, Beerse (BE); Regina DRIESEN, Beerse (BE); Filip VANHOUTTE, Beerse (BE); Annelien VAN DROOGENBROECK, Beerse (BE); Bernd VAN SNICK, Beerse (BE); Elise VAES, Beerse (BE)

(21) Appl. No.: 19/154,046

(22) PCT Filed: Feb. 6, 2024
(86) PCT No.: PCT/US2024/014585

§ 371 Date: Aug. 6, 2025

Related U.S. Application Data
(60) Provisional application No. 63/483,486, filed on Feb. 6, 2023.

(51) Int. Cl. A61K 9/28; A61K 9/00; A61K 9/16; A61K 9/20; A61K 31/513

(52) U.S. Cl. CPC A61K 9/2853; A61K 9/0053; A61K 9/1623; A61K 9/1652; A61K 9/2018; A61K 9/2054; A61K 9/2095; A61K 9/2813; A61K 9/282; A61K 9/284; A61K 31/513

(57) ABSTRACT

Solid pharmaceutical compositions of a FXIa inhibitor for oral administration are provided.

### CROSS-REFERENCE TO RELATED APPLICATION

This application claims the benefit of U.S. Provisional Application No. 63/483,486, filed Feb. 6, 2023, the entirety of which is incorporated by reference herein.

### TECHNICAL FIELD

The technical field of the present invention is in pharmaceuticals, particularly formulations of a FXIa inhibitor in oral solid dosage forms prepared by direct compression methods.

### BACKGROUND

Oral anticoagulants are a mainstay for prevention and treatment of venous and arterial thromboembolism. Although direct oral anticoagulants have replaced vitamin K antagonists for many indications, bleeding remains the major side effect. Fear of bleeding contributes to the underuse of anticoagulants in eligible patients with atrial fibrillation, and the inappropriate use of low dose direct oral anticoagulant regimens. (Steinberg et al., International trends in clinical characteristics and oral anticoagulation treatment for patients with atrial fibrillation: Results from the GARFIELD-AF, ORBIT-AF I. and ORBIT-AF II registries. Am Heart J 2017:194:132-40: Sanghai et al., Rates of potentially inappropriate dosing of direct-acting oral anticoagulants and associations with geriatric conditions among older patients with atrial fibrillation: The SAGE-AF study. J Am Heart Assoc 2020; 9:e014108). Therefore, a need for safer oral anticoagulants persists.

Factor XI is a promising target for development of new anticoagulants because it is an important driver of thrombus growth but plays a subsidiary part in hemostasis (Weitz et al., Factor XI inhibition to uncouple thrombosis from hemostasis: JACC review topic of the week. J Am Coll Cardiol 2021; 78:625-31).

Milvexian is a direct-acting, reversible, small molecule therapeutic agent that binds to and inhibits the activated form of human coagulation Factor XI (FXIa) with high affinity and selectivity. Milvexian is a macrocyclic compound having the structure of Formula (I):

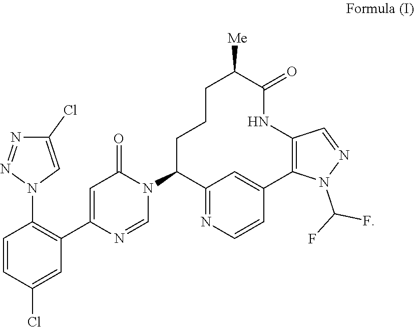 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

Milvexian has the chemical name (5R,9S)-9-(4-(5-chloro-2-(4-chloro-1H-1,2,3-triazol-1-yl)phenyl)-6-oxopyrimidin-1(6H)-yl)-21-(difluoromethvl)-5-methyl-21H-3-aza-1(4,2)-pyridina-2(5,4)-pyrazolacyclononaphan-4-one. Milvexian and a method of preparing milvexian are described in U.S. Pat. No. 9,453,018, which is hereby incorporated by reference in its entirety.

An amorphous solid dispersion (ASD) composition of milvexian in one or more polymers prepared by solvent-based spray drying methods, capsules thereof, and roller compaction tablets thereof prepared by dry granulation methods have been described in WO2020210629, which is hereby incorporated by reference in its entirety.

Tablets are composed of one or more active compounds and of tableting excipients, such as diluents, binders, lubricants and disintegrating agents. The active compound and the excipients are generally provided in the form of powders which are subjected to tableting, with or without preliminary treatment. There are currently three general methods of tablet preparation: (1) wet-granulation method, (2) dry-granulation method (slugging or roller compaction), and (3) direct compression (Williams et al., “Strategies to Address Low Drug Solubility in Discovery and Development”, 2013. Pharmacol. Rev., V65. pp. 215-499). All three methods have limitations.

Wet-granulation method is the most widely used method. Its popularity is due to the greater probability that the granules will meet all the physical requirements for the manufacture of good tablets. Its main limitations are the number of separate steps involved and the time and labor necessary to carry out the procedure. The steps involved in the wet-granulation method are: (1) weighing, (2) mixing, (3) granulation, (4) screening the damp mass after granulation, (5) drying (6) dry screening (7) lubrication, and (8) compression.

Dry-granulation method is generally used when tablet ingredients are sensitive to moisture or are unable to withstand elevated temperatures during drying. This method eliminates a number of steps but still includes (1) weighing. (2) mixing, (3) dry granulation, (4) dry screening, (5) lubrication, and (6) compression. However, this method requires that the tablet ingredients must have sufficient inherent binding or cohesive properties for dry granulation. Dry granulation is typically used for the manufacture of tablets comprising active compounds which are soluble in water or sensitive to heat and to moisture or have insufficient cohesive properties. This technique is less suited to low doses of active compounds because of the difficulty of obtaining homogeneous blends of dry powders.

Direct compression (DC) consists of compressing tablets directly from the tablet ingredients without wet or dry granulation. The advantages of direct compression include uniformity of blend, few manufacturing steps involved, i.e., the overall process involves only three steps: (1) weighing, (2) mixing, and (3) compression, hence makes possible a considerable saving in time; elimination of heat and moisture, prime particle dissociation and physical stability. However, direct compression is usually limited to those situations where the drug or active ingredient has the required physical and chemical properties. e.g., compactibility and low stickiness, to form pharmaceutically acceptable tablets. Given that the majority of active compounds have poor compressibility and/or are used in a low amount per unit dose, they have to be blended with excipients which are directly compressible and which are compatible with the active compound in order to be able to be subjected to direct compression.

Segregation could be another potential problem of the direct compression technique arises from the risk of separation of the powders or “demixing”. This demixing leads to tablets which are nonhomogeneous in composition. Thus, using the direct compression technique, it is possible to observe a poor distribution of the active compound in the excipients and a separation of the active compound and the excipients during the blending operation and in particular during all the transfer operations, leading to a variation in weight and in the content of active compound of the tablets. The poor flowability of the blend of powders generally is an aggravating factor. The separation of the active compound and excipients in the blend of powders before tableting is observed in particular when the active compound and excipients differ greatly in particle size.

A further potential problem of direct compression technique is the potential size of the compressed tablets. If the amount of active ingredient is high, a pharmaceutical formulator may choose to wet granulate the active ingredient with other excipients to attain an acceptable sized tablet with the desired amount of active ingredient. The amount of filler, binder or other excipients needed in wet granulation is less than that required for direct compression since the process of wet granulation contributes toward the desired physical properties of the tablet.

Moreover, for manufacturability reasons high speed rotary machines are used. In the direct compression process, the feed device, which generally operates by gravity, is very sensitive to the agglomeration of the powders or to the setting solid thereof. The rheology of the blend of powders to be tableted is therefore a determining factor in guaranteeing the uniformity in weight of the tablets and the uniformity of their contents. For products where large volumes are needed it is highly beneficial to produce the tablets using Continuous Manufacturing technology. In this context direct compression has distinct advantages over wet and dry Granulation methods due to the fewer and simpler steps. There are, however, distinct challenges to the properties of the active compound and the tablet ingredients, which needs to be optimized/selected to be feasible for continuous high speed manufacture.

Therefore, there is a need in the industry for techniques and pharmaceutical excipients which will allow manufacturers to prepare FXIa inhibitor tablets by direct compression by either batch manufacture or continuous manufacturing.

### Objectives

There exists a need for improved pharmaceutical formulations of active pharmaceutical ingredients (API), such as the FXIa inhibitors described in U.S. Pat. No. 9,453,018. In particular, there exists a need for pharmaceutical formulations with an acceptable bioavailability, in particular in solid dosage form prepared by direct compression processes. Moreover, there exists a need for pharmaceutical formulations with an acceptable bioavailability, in particular in solid dosage form prepared by direct compression processes that are amenable to continuous manufacturing.

WO2020210629 describes spray-dried amorphous solid dispersion (spray-dried powder; “SDP”)-based capsule formulations and roller compacted (RC) tablets formed by dry granulation of SDP containing milvexian and a Hydroxypropylmethylcellulose acetate succinate (HPMC-AS) in 3:1 weight ratio of milvexian to HPMC-AS. The RC tablets in this application showed a substantial drop in the dissolution profile compared to RC blend in the capsule.

An objective of the present invention is to provide a solid-state form of milvexian, or a pharmaceutically acceptable salt form thereof, that exhibits significantly improved solubility and bioavailability to the amorphous form, while maintaining acceptable physical and chemical stability.

An objective of the present invention is to provide amorphous solid dispersions of milvexian, or a pharmaceutically acceptable salt form thereof, that is kinetically stable according to regulatory requirements.

An objective of the present invention is to provide particles of spray-dried amorphous solid dispersion of milvexian in HPMC-AS-MG grade polymer to give improved physical stability, for example, improved performance with respect to particle brittleness compared to the spray dried solid dispersion particles described by WO2020210629.

An objective of the present invention is to provide a pharmaceutical powder blend that exhibits suitable physical properties amendable for direct compression tableting manufacture, for example, free flow property, compressibility, tap and bulk density, and particle sizes distribution.

An objective of the present invention is to provide a tablet manufactured by direct compression methods comprising spray-dried amorphous solid dispersion of milvexian in HPMC-AS-MG grade polymer in a weight ratio of 3:1 (milvexian: HPMC-AS-MG).

An objective of the present invention is to provide a direct compressed tablet comprising spray-dried amorphous solid dispersion of milvexian in HPMC-AS-MG grade polymer in a weight ratio of 3:1 (milvexian: HPMC-AS-MG) for which the exposure of milvexian better matches the exposure of the capsule formulation described by WO2020210629.

An objective of the present invention is to reduce the amount of excipients in solid dosage forms (e.g. tablets) of milvexian, or a pharmaceutically acceptable salt form thereof.

An objective of the present invention is to provide SDP formulations of HPMC-AS and milvexian, or a pharmaceutically acceptable salt form thereof, that has a high solubility in organic solvents or solvent mixtures.

An objective of the present invention is to provide SDP formulations of HPMC-AS and milvexian, or a pharmaceutically acceptable salt form thereof, that are suitable for processing by solvent evaporation to provide a powder suitable for direct compression into tablets.

An objective of the present invention is to provide SDP formulations of HPMC-AS and milvexian, or a pharmaceutically acceptable salt form thereof, that by proper solvents or solvent mixture selection results in a solution with properties suitable for solvent removal and solids formation.

An objective of the present invention is to provide SDP formulations of HPMC-AS and milvexian, or a pharmaceutically acceptable salt form thereof, where spray drying of the feed solution results in SDP particles with improved physical stability.

An objective of the present invention is to provide SDP formulations of HPMC-AS and milvexian, or a pharmaceutically acceptable salt form thereof, where spray drying of the feed solution results in SDP particles with improved downstream manufacturability as evidenced by improved compressibility (particle brittleness) and flowability.

An objective of the present invention is to provide SDP formulations of milvexian, or a pharmaceutically acceptable salt form thereof, where the spray-dried particles have improved dissolution rate or dissolution properties.

An objective of the present invention is to provide SDP formulations of milvexian, with a high content of milvexian to reduce the pill burden.

An objective of the present invention is to reduce the pill burden of patients treated with milvexian, or a pharmaceutically acceptable salt form thereof.

An objective of the present invention is to provide SDP formulations of milvexian, or a pharmaceutically acceptable salt form thereof, where spray drying of the feed solution results in SDP particles suitable for tabletting by Continuous Manufacturing methods.

An objective of the present invention is to provide tablet formulations of milvexian as SDP formulations of milvexian, or a pharmaceutically acceptable salt forms thereof, with a similar or improved exposure compared to the capsule formulation.

An objective of the present invention is to provide tablet formulations of milvexian as SDP formulations of milvexian, or a pharmaceutically acceptable salt forms thereof, that are suitable for manufacture by Continuous Manufacturing.

An objective of the present invention is to provide tablet formulations of milvexian as SDP formulations of milvexian, or a pharmaceutically acceptable salt forms thereof, that are suitable for Film Coating.

An objective of the present invention is to provide tablet formulations of milvexian as SDP formulations of milvexian, or a pharmaceutically acceptable salt forms thereof, that are chemically and physically compatible with coating.

An objective of the present invention is to provide tablet formulations of milvexian as SDP formulations of milvexian, or a pharmaceutically acceptable salt forms thereof, that exhibits low inter-patient variability at the clinical dose range (FIGS. 11A-B).

An objective of the present invention is to reduce the food effect on the bioavailability of milvexian comprised in tablets, or a pharmaceutically acceptable salt form thereof. See Example 5.

### SUMMARY

The disclosure provides solid pharmaceutical compositions for oral administration comprising: (a) a spray-dried amorphous solid dispersion (SDP) consisting essentially of milvexian free form and a pH-dependent enterosoluble polymer; (b) a binder that is microcrystalline cellulose (MCC), silicified microcrystalline cellulose (SMCC), or a combination thereof; (c) a filler that is lactose monohydrate; (d) a disintegrant; and (e) a lubricant: wherein milvexian free form is present in an amount ranging from about 10.0 wt. % to about 40.0 wt. % of the total weight of the solid pharmaceutical composition; and wherein the binder and lactose monohydrate are present in a weight ratio (binder:lactose monohydrate) ranging from about 3:2 to about 3:1.

The disclosure further provides pharmaceutical tablets comprising (1) a core comprising: (a) a spray-dried amorphous solid dispersion (SDP) consisting essentially of milvexian free form and a pH-dependent enterosoluble polymer; (b) a binder selected from microcrystalline cellulose (MCC), silicified microcrystalline cellulose (SMCC), or a combination thereof; (c) lactose monohydrate: (d) a disintegrant; (e) a lubricant; and (2) a film coating covering the core; wherein milvexian is present in an amount ranging from about 10.0 wt. % to about 40.0 wt. % of the total weight of the core; and wherein the binder and lactose monohydrate are present in the core in a weight ratio (binder:lactose monohydrate) ranging from about 3:2 to about 3:1.

The disclosure further provides spray-dried amorphous solid dispersions (SDP) consisting essentially of 75 wt. % milvexian and 25.0 wt. % of a pH-dependent enterosoluble polymer by the total weight of the SDP, wherein the SDP has a median particle size distribution of D_{v_50}≤60 μm with a span of about 1.9. In another embodiment, the SDP has a median particle size distribution of D_{v_50}≤50 μm with a span of about 1.9. In some embodiments, the SDP has a median particle size distribution of D_{v,50}≤45 μm with a span of about 1.9.

### BRIEF DESCRIPTION OF THE DRAWINGS

FIG. 1 shows a flow chart of the spray drying manufacture process for the spray-dried amorphous solid dispersion (SDP) of milvexian and HPMC-AS-MG in a weight ratio of 3:1.

FIG. 2. Illustrates the dissolution profiles for Ex.7 and Ex.8 by the dissolution test performed in 900 mL of dissolution medium at 37.0° C. using Paddle Apparatus (USP type 2, Ph.Eur., JP.) at a rotation speed of 75 rpm.

FIG. 3 shows the particle size distribution of Ex.8 (Tablettose®) in Example 2C.

FIG. 4 shows the particle size distribution of Ex.7 (Supertab 11SD) in Example 2C.

FIG. 5 shows dissolution profiles of Ex.9 (blue), 7 (red), and Ex.10 (green) in Example 2D by the quality control dissolution test performed in 900 mL of dissolution medium at 37.0° C. using Paddle Apparatus (USP type 2, Ph.Eur., JP.) at a rotation speed of 75 rpm.

FIG. 6 shows dissolution profiles of Ex.9 (blue), Ex.7 (red), and Ex.10 (green) in Example 2D by the dissolution test SGF→FaSSIF performed in 900 mL of dissolution medium at 37.0° C. using Paddle Apparatus (USP type 2, Ph.Eur., JP.) at a rotation speed of 75 rpm.

FIG. 7 shows the manufacturing process flow chart of milvexian 25 mg oral tablet core and 100 mg oral tablet core.

FIG. 8 shows the manufacturing process flow chart of milvexian 25 mg oral film-coated tablet and 100 mg oral film-coated tablet.

FIG. 9 shows the PXRD patterns for the SDPs produced in Example 1e. The PXRD patterns evidenced milvexian exists as amorphous form in the SDPs.

FIG. 10 shows the dissolution profiles of 25 mg and 100 mg film-coated tablets after 3 month storage at 40° C./75% RH by the quality control dissolution test performed in 900 mL of dissolution medium at 37.0° C. using Paddle Apparatus (USP type 2, Ph.Eur., JP.) at a rotation speed of 75 rpm.

FIG. 11A shows the milvexian plasma concentration as a function of time after administration of a film-coated direct compression tablet of the disclosure compared to the milvexian plasma concentration as a function of time after administration of a milvexian-containing capsule. See bioavailability study described in Example 5.

FIG. 11B shows the milvexian plasma concentration as a function of time after administration of a film-coated roller compaction tablet compared to the milvexian plasma concentration as a function of time after administration of a milvexian-containing capsule. See bioavailability study described in Example 5.

FIG. 12A shows the milvexian plasma concentration as a function of time after administration of a film-coated direct compression tablet of the disclosure to fasting patients compared to the milvexian plasma concentration as a function of time after administration of a film-coated direct compression tablet of the disclosure to fed patients. See food effect study described in Example 5.

FIG. 12B shows the milvexian plasma concentration as a function of time after administration of a film-coated roller compaction tablet to fasting patients compared to the milvexian plasma concentration as a function of time after administration of a film-coated roller compaction tablet to fed patients. See food effect study described in Example 5.

FIG. 13A shows the Day 1 milvexian plasma concentration as a function of time after BID administration of a film-coated direct compression tablet (2×100 mg) of the disclosure compared to the Day 1 milvexian plasma concentration as a function of time after BID administration of a milvexian-containing capsule (2×100 mg). See Example 5.

FIG. 13B shows the Day 5 milvexian plasma concentration as a function of time after BID administration of a film-coated direct compression tablet (2×100 mg) of the disclosure compared to the Day 5 milvexian plasma concentration as a function of time after BID administration of a milvexian-containing capsule (2×100 mg). See Example 5.

FIG. 13C shows the Day 1 milvexian plasma concentration as a function of time after BID administration of a film-coated direct compression tablet (1×25 mg) of the disclosure compared to the Day 1 milvexian plasma concentration as a function of time after BID administration of a milvexian-containing capsule (1×25 mg). See Example 5.

FIG. 13D shows the Day 5 milvexian plasma concentration as a function of time after BID administration of a film-coated direct compression tablet (1×25 mg) of the disclosure compared to the Day 5 milvexian plasma concentration as a function of time after BID administration of a milvexian-containing capsule (1×25 mg). See Example 5.

### DETAILED DESCRIPTION OF ILLUSTRATIVE EMBODIMENTS

### Definitions

The term “about” as used herein, refers to the actual given value, and it is also meant to refer to the approximation to such given value that would reasonably be inferred based on the ordinary skill in the art, including approximations due to the experimental and/or measurement conditions and acceptable error margins, for such given value. The term “about” as used herein refers to 0%, ±5.0%, ±10.0%, ±15.0%, ±20.0% or ±25.0% of a recited numeric value, inclusive. In a preferred embodiment, the term “about” as used herein refers to ±10.0% of a recited numeric value. For example, the phrase “about 8” refers to a value of 7.2 to 8.8, inclusive; as another example, the phrase “about 8%” refers to a value of 7.2% to 8.8%, inclusive.

Where present, all ranges are inclusive and combinable. For example, when a range of “1 to 5” is recited, the recited range should be construed as including ranges “1 to 5”, “1 to 4”, “1 to 3”, “1-2”, “1-2 and 4-5”, “1-3 and 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

The term “amorphous” refers to solids in which there is no long-range ordering of the molecules. The term amorphous also refers to solids comprising regions of crystallinity and regions that are amorphous. The term amorphous also encompasses semi-crystalline solids.

In the context of the present invention, the terms “amorphous solid dispersion” or “ASD” are used whereas in the literature some authors use the term solid solution which has the same meaning as solid dispersion in the context of the present invention. In the following, the different types of solid dispersions (solid solutions, glass solutions, glass suspensions, amorphous precipitations in a crystalline carrier, eutectics or monotecics, compound or complex formation and combinations thereof) are collectively referred to as solid dispersion.

In the context of the present invention, the term “crystallization” means crystallization as the active ingredient was not crystalline before and/or recrystallization as the active ingredient was crystalline, was then transferred in the amorphous form, stabilized in the amorphous form and does thereafter crystalize again.

The term “solid dispersion” defines a system in a solid state (as opposed to a liquid or gaseous state) comprising the components of the present compositions, wherein one component is dispersed more or less evenly throughout the other component or components (the components may include additional pharmaceutically acceptable formulating agents, generally known in the art, such as plasticizers, preservatives and the like). When said dispersion of the components is such that the system is chemically and physically uniform or homogenous throughout or consists of one phase as defined in thermodynamics, such a solid dispersion is called a “solid solution”. Solid solutions are preferred physical systems because the components therein are usually readily bioavailable to the organisms to which they are administered. This advantage can probably be explained by the ease with which said solid solutions can form liquid solutions when contacted with a liquid medium such as the gastrointestinal juices. The ease of dissolution may be attributed at least in part to the fact that the energy required for dissolution of the components from a solid solution is less than that required for the dissolution of components from a crystalline or microcrystalline solid phase.

The solid solution may be a continuous solid solution, in which milvexian, or a pharmaceutically acceptable salt form thereof, is molecularly dispersed throughout a matrix formed by the orally pharmaceutically acceptable polymer.

The solid solution may be a discontinuous solid solution, in which milvexian, or a pharmaceutically acceptable salt form thereof, is molecularly dispersed throughout a matrix formed by the orally pharmaceutically acceptable polymer. This discontinuous solid solution is partially miscible and presents two phases even though milvexian is molecularly dispersed.

The solid solution may be a substitutional solid solution, in which milvexian, or a pharmaceutically acceptable salt form thereof, is molecularly dispersed throughout a matrix formed by the orally pharmaceutically acceptable polymer. In this substitutional solid solution, the molecular diameter of milvexian differs less than 15% from the matrix (orally pharmaceutically acceptable polymer) diameter. In this case milvexian and matrix are substitutional. This substitutional solid solution can be continuous or discontinuous. When discontinuous, two phases are present even though milvexian is molecularly dispersed.

The solid solution may be an interstitial solid solution, in which milvexian, or a pharmaceutically acceptable salt form thereof, is molecularly dispersed throughout a matrix formed by the orally pharmaceutically acceptable polymer. In this interstitial solid solution, the molecular diameter of milvexian is less than 59% of the matrix (orally pharmaceutically acceptable polymer) diameter.

The term “solid dispersion” also comprises dispersions which are less homogenous throughout than solid solutions. Such dispersions are not chemically and physically uniform throughout or comprise more than one phase. For example, the term “solid dispersion” also relates to a system having domains or small regions wherein amorphous, microcrystalline or crystalline drug compound, and/or amorphous, microcrystalline or crystalline orally pharmaceutically acceptable polymer, and optionally amorphous, microcrystalline or crystalline surfactant, are dispersed more or less evenly in another phase comprising a solid solution comprising a drug compound, a polymer, and optionally a surfactant. Said domains are regions within the solid dispersion distinctively marked by some physical feature, small in size, and evenly and randomly distributed throughout the solid dispersion.

The term “flow” means that a bulk solid is deformed plastically due to the loads acting on it (e.g. failure of a previously consolidated bulk solid specimen). The magnitude of the load necessary for flow is a measure of flowability.

The term “flow function coefficient (FFC)” as used herein is frequently used to classify and compare powders as to their flowability (Svarovsky, 1987). A higher value of FFC indicates better flowability (see Table below). The phrase “good flow behavior” usually means that a bulk solid flows easily, i.e., it does not consolidate much and no flow promoting devices are required. Products are “poorly flowing” if they experience flow obstructions or consolidate during storage or transport.

| FFC value | Powder Flow Properties |
|---|---|
| 1 to 2 | Poor flowability (cohesive) |
| 2 to 4 | Moderate flowability |
| 4 to 10 | Good flowability |
| ≥10 | Excellent flowability (free flowing) |

The term “bulk density” is defined as the mass of the many particles of the material divided by the total volume they occupy. The total volume includes particle volume, inter-particle void volume, and internal pore volume. (“Powder Bulk Density-Bulk Solids density-Bulk Powder Properties-Powder Loose Density-Powder tapped density-PowderProcess.net”. www.powderprocess.net. Retrieved 2018-02-22.)

As used herein, the “tapped density” of a powder is the ratio of the mass of the powder to the volume occupied by the powder after it has been tapped for the predetermined number of taps in various methods. Tapped density can be calculated using the equation “Tapped Density (g/mL)=M/V_{f}”, where M=mass in grams, and V_{f}=the tapped volume in milliliters. The tapped density of a powder represents the most compact packing of the given powder”—according to the USP <616> one continue tapping until no further change in volume. The tapped and untapped (bulk) densities are determined by demarcating a small cuvette with known volumes, then inserting a small mass of powder into the cuvette (bulk density) and tapping it vertically against a padded bench top 50 times (tapped density). See Ferreira et al., Multivariate Analysis in the Pharmaceutical Industry, Academic Press, 2018, Chapter 10, pp. 235-267.

The term “Hausner ratio” refers to a ratio of tapped density to bulk density. It has been suggested that the Hausner ratio may provide an indication as to the flowability of a powder.

| Flowability Profile | Hausner Ratio |
|---|---|
| Excellent / Very Free Flow | 1.00-1.11 |
| Good / Free Flow | 1.12-1.18 |
| Fair | 1.19-1.25 |
| Passable | 1.26-1.34 |
| Poor Flow / Cohesive | 1.35-1.45 |
| Very Poor Flow / Very Cohesive | 1.46-1.59 |
| Approximatively no flow | >1.60 |

The term “pH-dependent enterosoluble polymer” denotes a polymer that is stable and does not dissolve in the stomach and the upper parts of the gastrointestinal tract, but readily dissolves when it arrives at the desired part of the gut to release the active pharmaceutical ingredient (API) contained therein. The solubility of a pH-dependent enterosoluble polymer depends on the conditions of acidity or alkalinity found all along the gut. In some embodiments, the SDP particles consist essentially of milvexian and pH-dependent enterosoluble polymer in a 3:1 weight ratio as described herein and mainly dissolve in the small intestine. In other embodiments, the SDP particles consist essentially of milvexian and pH-dependent enterosoluble polymer in a 3:1 weight ratio as described herein and will begin to dissolve in the small intestine. The solubility of the pH dependent enterosoluble polymer are measured in USP phosphate buffer according to the manufacturer's product brochure (https://www.setylose.com/fileadmin/download_pfmd/49.pdf), or solubility test described by Sarabu et al. in Hypromellose Acetate Succinate based Amorphous Solid Dispersions via Hot Melt Extrusion: Effect of Drug Physicochemical Properties, Carbohydr Polym. 2020 Apr. 1; 233: 115828.

The term “solid pharmaceutical composition for oral administration,” as used herein, encompasses pharmaceutical powder blends (such as those suitable for tableting or encapsuling), pharmaceutical powder blends for direct oral administration, as well as pharmaceutical dosage forms (e.g., tablets, capsules) made from such pharmaceutical powder blends.

The term “milvexian free form,” as used herein, refers to milvexian that is not in a salt form and not in a solvated form (i.e., non-solvated milvexian free form).

The term “aqueous medium,” as used herein, refers to a liquid medium that contains water. Aqueous media include water, fruit juices such as apple juice, vegetable juices, saline, buffer, and the like.

The terms “wt. %”. “% by weight”, and “% (w/w)”, as used herein, refer to the weight of the indicated ingredient as a percentage of the weight of the indicated composition. For example, if a composition is said to comprise 10.0 wt. % filler (or 10.0% by weight filler), then 10.0% of the weight of the composition is contributed by the filler. That is, for every 100 grams of composition, 10 grams is filler.

### Spray Dried Amorphous Solid Dispersion (SDP)

The pharmaceutical compositions described herein comprise an amorphous solid dispersion of milvexian free form.

In some embodiments, the pharmaceutical compositions of the disclosure comprise a spray-dried amorphous solid dispersion of mivexian free form.

The most frequent challenges in pharmaceutical development to prepare an amorphous solid dispersion (ASD) include, but are not limit to: (1) selection of an appropriate manufacturing technology; (2) the physical stability of the drug and the amorphous solid dispersion; (3) polymer matrix type and the amount of polymer matrix; (4) the ratio of API to the polymer matrix with balanced stability and satisfactory API release rate; and (5) physical and chemical stability of the amorphous solid dispersion and the incorporated API. (Williams et al., supra; Anane-Adjei et al., “Amorphous solid dispersions: Utilization and challenges in preclinical drug development within AstraZeneca”. 2022, Int. J. Pharm., V614+He and Ho, “Amorphous Solid Dispersions—Utilization and Challenges in Drug Discovery and Development”, 2015, J. Pharm Sci., V104, pp.3237-3258)

When developing an amorphous solid dispersion, an additional obstacle is that the challenges described have to be addressed simultaneously, interaction of challenges/choices (i.e. choice of polymer matrix can affect choice of manufacturing technology and stability, and vice versa) and based on limited information.

The physical properties of SDP particles prepared by spray drying process are dependent on a) API polymorphic form selection, b) the solvent system used in the manufacturing process, c) the polymer matrix used, and d) the manufacturing process. The methods and excipients chosen for the present invention are in some aspects in contrast to the methods and excipients known by the person skilled in the art and which are known as common to prepare an amorphous solid dispersion (ASD).

During development of the SDP for milvexian and HPMC-AS, different polymorphic forms of milvexian, different solvent systems for spray drying feed solutions were evaluated. Eventually, crystalline P1.acetone form of milvexian (acetone solvate) was selected together with dichloromethane (DCM)/MeOH 80/20 w/w % ratio as solvent system for spray drying to result in an amorphous solid dispersion of amorphous milvexian free form in HPMC-AS matrix.

### a) Milvexian Polymorphic Form Selection

Milvexian compound (API) can exist in several polymorphic forms with varying solubility in organic solvents and stability, for example, amorphous form; crystalline forms of milvexian free form (e.g., Form A, Form B. Form C, Form D, Form E, Form F, Form H. Form I and Form J); and crystalline form of acetone solvate (P1.acetone). In principle, any of these polymorphic forms or solvates can be used to prepare the SDP. Preferred polymorphic forms or solvates are those that are most soluble in solvents that are useful for spray drying.

A crystalline acetone solvate form (P1.acetone) of milvexian may be prepared, for example, according to the process set forth in international patent application WO2022081473.

The amorphous form of milvexian may be prepared, for example, according to the spray drying procedures of Example 1 as described in WO 2020210629, which is incorporated herein by reference in its entirety. The amorphous form milvexian was characterized by an X-ray powder diffraction patterns showing no crystalline peaks. The amorphous form of milvexian was also characterized by modulated differential scanning calorimetry (mDSC) comprising an endotherm with an onset temperature of 160° C., and a peak glass transition temperature at about 163° C.

The crystalline forms of milvexian free form (e.g., Form A, Form B, Form C, Form D, Form E, Form F, Form H, Form I and Form J) may be prepared, for example, according to the procedures of Example 1 as described in WO 2021207659, which is incorporated herein by reference in its entirety. For example, crystalline Form A of milvexian free form has an X-ray powder diffraction pattern (CuKα λ=1.541874 Å at room temperature) comprising at least one peak selected from 5.0, 5.3, 8.2, 10.0, 10.7, 10.9, 13.0, 14.6, 15.1, 161.1, 17.2, 190.0, 19.5, 20.5, 21.5, 22.9 and 24.5 degrees two theta±0.2 degrees two theta, wherein the PXRD pattern of Form J is measured at room temperature. Crystalline Form J of milvexian free form has an X-ray powder diffraction pattern (CuKα λ=1.541874 Å at room temperature) comprising at least one peak selected from 7.4±0.2, 9.0±0.2, 11.3±0.2, 11.7±0.2, 12.5±0.2, 14.8±0.2, 15.1±0.2, 15.5±0.2, 16.1±0.2, 17.6±0.2, 18.4±0.2, 18.8±0.2, 19.3±0.2, 23.3±0.2, 24.4±0.2, 26.2±0.2, 26.6±0.2, 27.7±0.2, 28.2±0.2, and 28.8±0.2 degrees two theta, wherein the PXRD pattern of Form J is measured at room temperature. Crystalline forms A-J may also be characterized by solid state Nuclear magnetic resonance spectroscopy (ssNMR), infrared spectroscopy (IR), differential scanning calorimetry (DSC), dynamic vapor sorption (DVS), and thermogravimetric analysis (TGA).

Another crystalline form of milvexian acetone solvate (milvexian to acetone in 1:1 molar ratio, P1.acetone) may be prepared according to the procedures of Example 1 described in WO 2022081473, which is incorporated herein by reference in its entirety. The P1.acetone form has an X-ray powder diffraction pattern comprising at least one peak selected from 8,2321, 10,0872, 14,3163, 16,1898, 16,5524, 17,5729, 18,6786, 19,1386, 19,4389, 19,5888, 20,0236, 21,2896, 21,5821, 22,0945; 22,4947, 23,5085, 23,8930, 24,9851, 25,0767, 25,4275, 25,7772, 26,4620, 26,6794, 27,1315, 27,3484, 28,9275, 29,8608, 30,2353, 30,5195, 30,7433, 31,1200, 31,5951, 31,9657, 32,7989, 33,5411, 34,0682, 34,3433, 34,6898, 35,2079, 35,6653, 36,4135, 36,7245, 38,9401, 40,1133, 43,2735, 43,4015, 43,7011, 44,9886, 46,1717, 48,4294, 49,2681 degrees two theta±0.2 degrees two theta. Crystalline P1.acetone form may also be characterized by infrared spectroscopy (IR).

It was found that P1.acetone form is one of the most stable polymorphs. Typically, milvexian free form is preferred for spray-drying methods due to the absence of solvents. In some embodiments, the crystalline form of acetone solvate of milvexian (P1.acetone) prepared according to WO 2022081473, or crystalline Form J prepared according to WO 2021207659 is preferred as the starting material for the manufacturing of the spray-dried amorphous solid dispersion instead of amorphous or other known crystalline forms of milvexian, due to its superior morphology and reliable crystallization process. Other polymorphs with sufficient solubility in organic solvent system can also be used as starting material for preparing SDP, for example, crystalline Form J of milvexian free form with a solubility of 230 mg/mL in 80/20 w/w % DCM/MeOH, and crystalline Form A of milvexian free form with a solubility of 255 mg/mL in 80/20 w/w % DCM/MeOH.

### b) Solvent System Selection For Feed Solution Preparation

In an early effort to screen solvent system, aqueous solvent systems such as mixture of acetone and water (acetone/water) in various ratios (w/w %) was identified as effective to prepare the feed solution of milvexian and HPMC-AS polymer for spray drying process. It was found that, in a solvent system of acetone/water (90/10 w/w %), the solubility of milvexian is at 36 mg/mL at room temperature and 70 mg/mL at 49° C. The feed solution of milvexian as described above demonstrated chemical and physical stability for at least two weeks at 50° C.

Surprisingly, it was found that the organic solvent system comprising the combination of dichloromethane (DCM) and methanol in the ratio of 80 wt. % to 20 wt. % respectively, led to more than a 5-fold increase in milvexian solubility i.e. >200 mg/mL at room temperature for various polymorphic forms of milvexian. This increase in solubility combined with the chosen weight ratio of 3-to-1 of milveixan to HPMC-AS made it possible to achieve at least 15.0 wt. % of solute content (dry weight of HPMC-AS and milvexian by the total weight of the feed solution) in the feed solution at room temperature. This concentration is substantially higher than that typically used in ASD spray drying feed solutions. The feed solution of milvexian and HPMC-AS dissolved in an organic solvent system of DCM/MeOH (80/20 w/w %) is stable up to 14 days at room temperature and the viscosity is sufficiently high to enable formation of large particles suitable for direct compression. The increase in milvexian solubility enabled high solute content, which when spray dried resulted in a directly compressible SDP powders with good flowability and good bulk/tapped density. (See “Spray drying formulation of amorphous solid dispersions”, Singh and Van den Mooter, 2016, Advanced Drug Delivery Reviews, V100, pp.27-50. “Pharmaceutical amorphous solid dispersion: A review of manufacturing strategies”. Bhujbal et al., 2021, Acta Pharmaceutica Sinica B, V11(8), pp. 2506-2536. “Efficient production of solid dispersions by spray drying solutions of high solid content using a 3-fluid nozzle”, Kauppinen et al. 2018, European J of Pharm and Biopharm, V123, pp. 50-58). See Example 1.

In one embodiment, a feed solution having 15 wt. % solute content (dry weight of milvexian and HPMC-AS by the total weight of the feed solution) in 80/20 w/w % DCM/Methanol mixture was spray dried to provide a powder comprising micron particles having a median particle size of 38 μm, span of 1.67, bulk/tapped density 0.31/0.38 g/mL and a flow function coefficient of 9.3 indicating that the powder is easily flowable and almost free flowing (flow function coefficient (FFC) >10). This is a significant improvement over previous powders which had FFC in the range 4 to 5. The increased particle size, bulk/tapped density and highly improved flowability means that the SDP powder of milvexian/HPMC-AS (3:1 weight ratio) can be directly blended with tablet excipients and directly compressed, and in continuous manufacturing be directly fed to the blender without preprocessing or preblending with a flowability agent.

The DCM/Methanol solvent mixture is superior to the Acetone/Water solvent mixture due to higher API solubility at room temperature.

The methods described herein for preparing the SDPs do not result in detectable amounts impurities such as, e.g., 2-methoxy-1-propene and 2,2-dimethoxy propane.

c) pH-Dependent Enterosoluble Polymer Selection

In some aspects, the disclosure is directed to an amorphous solid dispersion (ASD) consisting essentially of milvexian and a pH-dependent enterosoluble polymer. In some embodiments, the disclosure is directed to a SDP comprising milvexian and a pH-dependent enterosoluble polymer.

In an embodiment, this disclosure provides particles of SDP of milvexian in HPMC-AS polymer with improved physical properties that are amenable for direct compression tablet manufacture processes, for example, free flow property measured by large particle size, bulk and tap density, and flow function coefficient, and improved particle brittleness/compressibility.

In some embodiments, this disclosure provides particles of SDP of milvexian and a pH-dependent enterosoluble polymer. In some embodiments, the SDP particles can be further combined with pharmaceutically acceptable excipients such as binders, fillers, diluents, flavors, colorant, lubricants, glidants, taste masking agent, preservatives, sorbents or sweeteners.

In some embodiments of the SDP particles of the disclosure, the pH-dependent enterosoluble polymer is selected from cellulose acetate trimellitate (CAT), cellulose acetate phthalate (CAP), Hydroxypropyl methylcellulose phthalate (HPMCP), Hydroxypropylmethylcellulose acetate succinate (HPMC-AS) LF, LG, MF, MG or HF Grades such as Agoat®, Polyvinyl acetate phthalate (PVAP) such as Sureteric® and Opadry® and Shellac resins such as SSB® Aquagold, or polyvinylpyrrolidone (PVP).

In some embodiments, the pH-dependent enterosoluble polymer is soluble in an aqueous medium at a pH of from about 5.5 to about 6.8, such as, for example, at a pH of about 5.5, about 5.6, about 5.7, about 5.8, about 5.9, about 6.0, about 6.1, about 6.2, about 6.3, about 6.4, about 6.5, about 6.6, about 6.7, or about 6.8.

In a preferred embodiment of the SDP described herein, the pH dependent enterosoluble polymer is soluble in an aqueous medium at a pH of greater than or equal to 6.0, such as, for example, a pH of 6.0, 6.1, 6.2, 6.3, 6.4, 6.5, 6.6, 6.7, 6.8, and the like.

In a preferred embodiment of the SDP described herein, the pH dependent enterosoluble polymer that is soluble in an aqueous medium at a pH of greater than or equal to 6.0 is selected from HPMC-AS, PVP, or any combination thereof.

In a preferred embodiment of the SDP described herein, the entersoluble polymer that is soluble in an aqueous medium at a pH of greater than or equal to 6.0 is selected from HPMC-AS-MG, HPMC-AS-LG.

In a more preferred embodiment of the SDP described herein, the pH dependent enterosoluble polymer that is soluble in an aqueous medium at a pH of greater than or equal to 6.0 is HPMC-AS.

In a further preferred embodiment, the HPMC-AS is selected from HPMC-AS-LG, HPMC-AS-MG. HPMC-AS-HG, HPMC-AS-LF. HPMC-AS-MF, HPMC-AS-HF. HPMC-AS-LMP, HPMC-AS-MMP, HPMC-AS-HMP, Affinisol™ HPMC-AS 716, Affinisol™ HPMC-AS 912, or Affinisol™ HPMC-AS 126.

In a further preferred embodiment, the HPMC-AS is selected from HPMC-AS-LG, HPMC-AS-MG, or HPMC-AS-HG.

In a most preferred embodiment, the HPMC-AS is HPMC-AS-MG.

In some embodiments of the SDP as described herein, milvexian free form and the pH dependent enterosoluble polymer are present in any weight ratio (w/w; milvexian free form:pH-dependent enterosoluble polymer).

In some embodiments of the SDP as described herein, milvexian free form and the pH dependent enterosoluble polymer are present in a weight ratio (w/w; milvexian:pH-dependent enterosoluble polymer) selected from about 1:1, about 1:3, or about 3:1.

In some embodiments of the SDP as described herein, milvexian free form and the pH dependent enterosoluble polymer are present in a weight ratio (w/w; milvexian:pH-dependent enterosoluble polymer) selected from 1:1, 1:3, or 3:1.

In a preferred embodiment of the SDP as described herein, milvexian free form and the pH dependent enterosoluble polymer are present in a weight ratio of about 3:1.

In another preferred embodiment of the SDP as described herein, milvexian free form and the pH dependent enterosoluble polymer are present in a weight ratio of 3:1.

In a preferred embodiment of the SDP as described herein, milvexian free form and the pH dependent enterosoluble polymer that is soluble in an aqueous medium at a pH of greater than or equal to 6.0 are present at a weight ratio (w/w; milvexian:pH-dependent enterosoluble polymer) selected from about 1:1, about 1:3 or about 3:1.

In another preferred embodiment of the SDP as described herein, milvexian free form and the pH dependent enterosoluble polymer that is soluble in an aqueous medium at a pH of greater than or equal to 6.0 are present at a weight ratio (w/w; milvexian:pH-dependent enterosoluble polymer) selected from 1:1, 1:3 or 3:1.

In another preferred embodiment of the SDP as described herein, milvexian free form and the pH dependent enterosoluble polymer that is soluble in an aqueous medium at a pH of greater than or equal to 6.0 are present at a weight ratio (w/w; milvexian:pH-dependent enterosoluble polymer) of about 3:1.

In another preferred embodiment of the SDP as described herein, milvexian free form and the pH dependent enterosoluble polymer that is soluble in an aqueous medium at a pH of greater than or equal to 6.0 are present at a weight ratio (w/w; milvexian:pH-dependent enterosoluble polymer) of 3:1.

In a more preferred embodiment of the SDP as described herein, milvexian free form and HPMC-AS MG are present in a weight ratio (w/w; milvexian:polymer) selected from about 1:1, about 1:3, or about 3:1.

In another more preferred embodiment of the SDP as described herein, milvexian free form and HPMC-AS MG are present in a weight ratio (w/w; milvexian:polymer) selected from 1:1, 1:3, or 3:1.

In a more preferred embodiment of the SDP as described herein, milvexian free form and HPMC-AS MG are present in a weight ratio (w/w; milvexian:polymer) of about 3:1.

In another more preferred embodiment of the SDP as described herein, milvexian free form and HPMC-AS MG are present in a weight ratio (w/w; milvexian:polymer) of 3:1.

In some embodiments, this disclosure provides SDP prepared by the method described herein have the compositions as in Table 1 below.

TABLE 1
75% milvexian:HPMC-AS-M SDP

Amount

| Component | wt. % | Amount per batch (g) |
|---|---|---|
| Milvexian free form* | 75 | 750 |
| HPMAC-AS MG | 25 | 250 |
| Total | 100 | 1000 |

*The API before spray drying is P1.actone solvate crystalline form of milvexian.
Acetone was removed by the spray drying process.

HPMC-AS or hydroxypropyl methylcellulose acetate succinate or hvpromellose acetate succinate is a mixture of acetic acid and monosuccinic acid esters of hydroxypropylmethyl cellulose (IUPAC name: cellulose, 2-hydroxypropyl methyl ether, acetate, hydrogen butanedioate). HPMC AS is an enterosoluble polymer commercially available in three grades; LG, MG, and HG. The key properties of HPMC AS are high Tg (119° C.-122° C.), amphiphilic nature, and insolubility in water and simulated gastric fluid, melt viscosity values are about 2.4-3.6 mPa·S. Different grades are available differentiated based on degree/ratio of substitution (acetyl content, succinoyl content) and particle size (micronized or fine (F), and granular (G)). Because HPMC-AS is dissolved in preparing the amorphous solid dispersions of the present invention, the particle size (F or G) is less relevant. The HPMC-AS grades are named differently depending on the manufacturer. HPMC-AS (AQOAT) was purchased from Shin-Etsu Chemical Co., Ltd. (Tokyo, Japan). Several grades of HPMC-AS are available. The manufacturer reports that the respective grades are soluble in McIlvaine's buffer solution at the following pH values: -LF and -LG. g 5.5: -MF and -MG, g 6.0; and —HF and -HG, g 6.8. However, we have found that the -LF and -LG grades are sparingly soluble, dispersing to form colloidal solutions in aqueous solutions at pH >about 4.8, the -MF and -MG grades at pH >about 5.2, and —HF and -HG grades at pH >about 5.7. (See Sarabu et al. supra).

For example, the AQOAT® brand of HPMC-AS by Shin-Etsu Chemical Co, Ltd. defines these grades as in Table 2 below:

TABLE 2
Chemical Properties of AQOAT ® brand HPMC-AS

| | | | | | | MW * | | | Mean |
|---|---|---|---|---|---|---|---|---|---|
| | | | | | | (Mn**, | Tg | pH | particle |
| Grade | | Acetyl % | | Succinoyl % | | Da) | (° C.) | solubility | size |
| Micronized | AS-LF | 8 | | 15 | | 12933 ± | | ≥5.5 | 5 μm |
| | | | | | | 666 | | | |
| | AS-MF | 9 | | 11 | | 12733 ± | | ≥6.0 | |
| | | | | | | 208 | | | |
| | AS-HF | 12 | | 6 | | 13033 ± | | ≥6.5 | |
| | | | | | | 306 | | | |
| Granular | AS-LG | 5-9 | (8) | 14-18 | (15) | 13267 ± | 119 | 5.5-6 | 1 mm |
| | | | | | | 503 | | | |
| | AS-MG | 7-11 | (9) | 10-14 | (11) | 13033 ± | 120 | 6-6.5 | |
| | | | | | | 351 | | | |
| | AS-HG | 10-14 | (12) | 4-8 | (6) | 12967 ± | 122 | >6.8 | |
| | | | | | | 58 | | | |

MW * molecular weight data for AQOAT@ brand HPMC-AS polymers are obtained from Fukasawa et al., Chem. Phar,. Bull, 2004, vol. 52, pp. 1391-1393,
Mn** number average molecular weight

Further grades of Shin-Etsu include AQOAT® HPMC-AS HPMC-AS-LMP, HPMC-AS-MMP, and HPMC-AS-HMP, having a medium particle size from about 70 to about 300 pm.

Dow® defines the grades of HPMC-AS with the brand Affinisol™ and a numeric code as shown in Table 3.

TABLE 3
Chemical Properties of Affinisol ™ brand HPMC-AS

| | Affinisol ™ | Affinisol ™ | Affinisol ™ |
|---|---|---|---|
| Grade | HPMC-AS 716 | HPMC-AS 912 | HPMC-AS 126 |
| hydroxylpropyl | 5.0%-9.0% | 5.0%-9.0% | 6.0%-10.0% |
| methoxyl | 20.0%-24.0% | 21.0%-25.0% | 22.0%-26.0% |
| Acetate substitution | 5.0%-9.0% | 7.0%-11.0% | 10.0%-14.0% |
| Succinate substitution | 14.0-18.0% | 10.0%-14.0% | 4.0%-8.0% |
| Viscosity* | 2.4-3.6 cP | 2.4-3.6 cP | 2.4-3.6 cP |

*Viscosity determined as a 2% solution in NaOH solution

Therefore, the HPMC-AS, in the amorphous solid dispersions with milvexian, may be selected from, and without being limited to, HPMC-AS-LG, HPMC-AS-MG, HPMC-AS-HG, HPMC-AS-LF, HPMC-AS-MF, HPMC-AS-HF, HPMC-AS-LMP, HPMC-AS-MMP, HPMC-AS-HMP grades as described in Table 2 above; Affinisol™ HPMC-AS 716, Affinisol™ HPMC-AS 912, and Affinisol™ HPMC-AS 126 grades as described in Table 3 above.

### d) Manufacturing Process Selection

The choice of manufacturing technology for an amorphous solid dispersion depends on several factors and considerations, including the following:
API melting point
Polymer glass transition temperature (T_{g})
Degradation temperatures for API and Polymer
API solubility in organic/aqueous media for solvent based manufacture process
API crystallization mechanism
Polymer solubility in organic/aqueous media for solvent based manufacturing process

The SDPs of the disclosure may be prepared using any suitable method. In some embodiments, the SDP of the disclosure is prepared by spray-drying (“spray-dried SDP”).

Here, the milvexian API melting point is generally 248° C. (depending on the specific polymorph) and, if present as an API-solvate, desolvation temperature was ca. 180° C. As most polymers degrade in the temperature range 175-250° C. manufacturing techniques based on melting weren't feasible due to the high processing temperatures. Hence, manufacturing techniques were limited to solvent-based techniques.

In some aspects, the SDP disclosed herein are prepared by spray drying a solution containing milvexian, a pH dependent enterosoluble polymer, and organic solvent(s).

In some embodiments, the SDP disclosed herein are prepared by spray drying a solution formed by dissolving milvexian and a pH dependent enterosoluble polymer in a mixture of dichoromethane/methanol.

In some embodiments, the SDP disclosed herein are prepared by spray drying a solution formed by dissolving milvexian P1.acetone crystalline form and a pH dependent enterosoluble polymer in a mixture of dichoromethane/methanol.

In some embodiments, the SDP disclosed herein are prepared by spray drying a solution formed by dissolving milvexian P1.acetone crystalline form and hypromellose acetate succinate (HPMC-AS) in a mixture of dichoromethane/methanol.

In some embodiments, the SDP disclosed herein are prepared by spray drying a solution formed by dissolving milvexian P1.acetone crystalline form and hypromellose acetate succinate (HPMC-AS) in a mixture of dichoromethane/methanol (80/20 w/w %).

In some embodiments, the SDP disclosed herein are prepared by spray drying a solution formed by dissolving a 3:1 w/w ratio (on a milvexian free-form basis) of milvexian P1.acetone crystalline form and hypromellose acetate succinate (HPMC-AS) in a mixture of dichoromethane/methanol (80/20 w/w %).

In embodiments in which a solvate of milvexian is used as the API source, such as the acetone in P1.acetone crystalline form, the solvent contributed by the solvate is removed during the spray-drying process such that the milvexian in the ASD is a free form.

In some aspects, the process of making the amorphous solid dispersion comprises (i) dissolving milvexian acetone solvate and HPMC-AS MG in a mixture of 80/20 (w/w) DCM/MeOH at a dissolved solids content of 15 wt. %, and (ii) spray drying the solution by feeding the solution at 20° C., to produce large droplet size.

### SDP Properties

The SDP of milvexian should have suitable characteristics for continuous feeding and manufacturability, such as good flowability. Flowability of the powder can be influenced by cohesiveness, hygroscopicity, specific surface area, particle size.

SDP prepared by the method as described herein have good flowability, good compressibility, and low sticking propensity; properties which are important for solids handling and tabletability. Moreover, the ASDs prepared herein are compatible with a direct compression method for preparing tablets. The spray drying methods disclosed herein also result in ASDs having particle sizes that contribute to these properties.

In some embodiments, the SDP is a solid solution of polymer in milvexian.

In some embodiments, the SDP has a particle size distribution of D_{v,50}≤60 mm with a span of about 1.9. In some embodiments, the SDP has a particle size distribution of D_{v,50}≤50 mm with a span of about 1.9. In some embodiments, the SDP has a particle size distribution of D_{v,50}≤45 mm with a span of about 1.9. In some embodiments, the SDP has a particle size distribution of D_{v,10}≤15 mm: D_{v,50}≤45 mm; and D_{v,50}≤95 mm. In a preferred embodiment, the SDP has a particle size distribution of D_{v,10}≤20 mm; D_{v,50}≤50 mm; and D_{v,90}≤110 mm. In a further preferred embodiment, the SDP has a particle size distribution of D_{v,10}≤25 mm; D_{v,50}≤60 mm; and D_{v,50}≤140 mm. In a further preferred embodiment, the SDP has a median particle size distribution of D_{v,50} 45 μm. In a most preferred embodiment, the SDP has a median particle size distribution of D_{v,50} of about 40 μm.

In some embodiments, the SDP has a bulk density of about 0.27 g/cm³ to about 0.36 g/cm³.

In other embodiments, the SDP has a tapped density of about 0.34 g/cm³ to about 0.45 g/cm³.

In some aspects, the SDP has an XRD that is consistent with the milvexian in the amorphous solid dispersion being amorphous, such as, for example, 100% amorphous, 99% amorphous, 98% amorphous, 97% amorphous, 96% amorphous, 95% amorphous, 94% amorphous, 93% amorphous, 92% amorphous, 91% amorphous, or 90% amorphous.

In some aspects, the SDP is stable with respect to the amorphous milvexian content. That is, the milvexian in the amorphous solid dispersion remains amorphous for an extended period of time, under a variety of conditions.

### Solid Pharmaceutical Compositions For Oral Administration

The solid pharmaceutical composition for oral administration of the disclosure encompasses pharmaceutical powder blends (such as those suitable for tableting or capsuling), pharmaceutical powder blends for direct oral administration, as well as unitary pharmaceutical dosage forms (e.g., tablets, capsules) made from such pharmaceutical powder blends.

a) Excipients used in Pharmaceutical Compositions of the Disclosure

In some aspects, the disclosure is directed to solid pharmaceutical compositions for oral administration comprising a spray-dried amorphous solid dispersion (SDP) consisting of milvexian free form and a pH-dependent enterosoluble polymer, and one or more pharmaceutically acceptable excipients selected from binder, filler, diluent, disintegrant, colorant, lubricant, glidant, and coating.

In some embodiments, the disclosure is directed to solid pharmaceutical compositions for oral administration comprising a spray dried amorphous solid dispersion (SDP) consisting essentially of milvexian free form and a pH-dependent enterosoluble polymer in a weight ratio (milvexian free form:pH-dependent enterosoluble polymer) of about 3:1, a binder selected from microcrystalline cellulose (MCC), silicified microcrystalline cellulose (SMCC), or a combination thereof; a filler that is lactose monohydrate; a disintegrant; and a lubricant; wherein milvexian free form is present in an amount ranging from about 10.0 wt. % to about 40.0 wt. % of the total weight of the solid pharmaceutical composition; and wherein the binder and lactose monohydrate are present in a weight ratio (binder:lactose monohydrate) ranging from about 3:2 to about 3:1.

In some embodiments, this disclosure provides solid pharmaceutical compositions for oral administration comprising SDP particles consisting essentially of milvexian and HPMC-AS-MG in a weight ratio of 3:1 (milvexian free form:HPMC-AS-MG), and one or more pharmaceutically acceptable excipients selected from binder, filler, diluent, disintegrant, colorant, lubricant, glidant, and coating.

In some embodiments, the solid pharmaceutical composition for oral administration is a tablet core, formed by direct compression of a pharmaceutical powder.

In some embodiments, the solid pharmaceutical composition for oral administration is a pharmaceutical powder blend, such as a powder blend suitable for tableting by direct compression.

In some embodiments, the pharmaceutical powder of the disclosure has properties which render the powder suitable for use in direct compression tableting.

In some embodiments, the pharmaceutical powder of the disclosure has a tap density of about 0.56 g/mL.

In some embodiments, the pharmaceutical powder of the disclosure has a bulk density of about 0.47 g/mL.

In some embodiments, the pharmaceutical powder of the disclosure has a Flow function coefficient (ring shear) of 10.28.

In some embodiments, the pharmaceutical powder of the disclosure has a Flow function coefficient (ring shear) of about 10.28.

In some embodiments, the pharmaceutical powder blend has the composition as in Table 4 below.

TABLE 4

Amount

| | | Amount | Amount |
|---|---|---|---|
| | | per dose | per dose |
| Component | wt. % | (25 mg) | (100 mg) |
| spray-dried amorphous solid | 22.22 | 33.33 | 133.3 |
| dispersion of milvexian and | | | |
| HPMC-AS-MG in a weight-by- | | | |
| weight ratio of 3:1 | | | |
| Silicified Microcrystalline | 43.07 | 64.6 | 258.4 |
| cellulose | | | |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Powder Blend Weight | | 150 | 600 |

The pharmaceutical powder blend as described in the table above is for the preparation of direct compressed immediate release oral tablet core containing silicified microcrystalline cellulose and lactose monohydrate as binders/fillers, croscarmellose sodium as disintegrant and magnesium stearate as lubricant. The powder blend of spray dried ASD with excipients of the tablet has been modified to optimize the tablet manufacturability.

In some embodiments, the present disclosure provides solid pharmaceutical compositions that are tablets having the compositions as in Table 5 below.

TABLE 5
spray-dried amorphous solid dispersion of milvexian and HPMC-AS-
MG in a weight-by-weight ratio of 3:1
Silicified Microcrystalline cellulose (SMCC 90)
Lactose monohydrate
Croscarmellose sodium
Magnesium stearate
wherein, the pharmaceutical tablet is further optionally coated with a film.

In some embodiments, the present disclosure is directed to solid pharmaceutical compositions that are immediate release, tablet cores having the following compositions as in Table 6 below.

TABLE 6
Immediate release Tablet Core

| Component | wt. % range | wt. % | Function |
|---|---|---|---|
| Drug load (DL) | 10-40, | 16.67 | API |
| | preferably | | |
| | 11-21 | | |
| Spray-dried amorphous solid | 13.3-53.3, | 22.22 | SDP |
| dispersion of milvexian and | preferably | | |
| HPMC-AS-MG in a weight- | 14.67-28.00 | | |
| by-weight ratio of 3:1 | | | |
| Silicified Microcrystalline | 21-71, | 43.07 | Binder |
| cellulose (SMCC 90) | preferably 31- | | |
| | 61, most | | |
| | preferred 38-48 | | |
| Lactose monohydrate | 25.0-33.0 | 28.71 | Filler |
| SMCC 90 to Lactose | 66/33 to 100/0 | 60/40 | |
| monohydrate weight | | | |
| ratio | | | |
| Croscarmellose sodium | 3.0-7.0 | 5.0 | Disintegrant |
| Magnesium stearate | 0-3; preferably | 1.0 | Lubricant |
| | 0.5-2.0; most | | |
| | preferably 0.5-1.5 | | |
| Total weight percent | 100 | 100 | |

wherein, the tablet core is further optionally coated with a film.

It has been found that the drug load affects powder blend flowability and the friability of the direct compression tablets (e.g., less than 1%). The workable drug load ranges from about 10.0 wt. % to about 40.0 wt. % based on the total weight of the solid pharmaceutical composition (powder blend or uncoated tablet). The preferred range for drug load ranges from about 11.0 wt. % to about 21.0 wt. % based on the total weight of the solid pharmaceutical compositions (powder blend or uncoated tablet). The most preferred drug load is 16.67 wt. % based on the total weight of the solid pharmaceutical compositions (powder blend or uncoated tablet).

In some aspects, the solid pharmaceutical compositions (powder blend or uncoated tablet) of the disclosure comprises about 10.0 wt. %-about 40.0 wt. % of milvexian free from based on the total weight of the solid pharmaceutical compositions (powder blend or uncoated tablet), such as, for example, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, or about 40.0 wt. % of milvexian free form.

In some embodiments, the solid pharmaceutical compositions (powder blend or uncoated tablet) of the disclosure comprises about 15.0 wt. % to about 28.0 wt. % of milvexian free form based on the total weight of the solid pharmaceutical compositions (powder blend or uncoated tablet), such as, for example, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, or about 28.0 wt. %, of milvexian free form.

In other embodiments, the solid pharmaceutical compositions (powder blend or uncoated tablet) of the disclosure comprises about 11.0 wt. % to about 21.0 wt. % of milvexian free form based on the total weight of the uncoated tablet, such as, for example, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, or about 21.0 wt. %, of milvexian free form.

In some embodiments, the solid pharmaceutical compositions (powder blend or uncoated tablet) of the disclosure comprises about 16.67 wt. % of milvexian free form based on the total weight of the solid pharmaceutical compositions (powder blend or uncoated tablet).

In some aspects, the solid pharmaceutical compositions (powder blend or uncoated tablet) of the disclosure comprises about 13.3 wt. % to about 53.3 wt. % of the SPD consisting essentially of milvexian free form and a pH-dependent enterosoluble polymer in a weight ratio (milvexian free form:pH-dependent enterosoluble polymer) of about 3:1, such as for example, about 13.3 wt. %, about 14.3 wt. %, about 15.3 wt. %, about 16.3 wt. %, about 17.3 wt. %, about 18.3 wt. %, about 19.3 wt. %, about 20.3 wt. %, about 21.3 wt. %, about 22.3 wt. %, about 23.3 wt. %, about 24.3 wt. %, about 25.3 wt. %, about 26.3 wt. %, about 27.3 wt. %, about 28.3 wt. %, about 29.3 wt. %, about 30.3 wt. %, about 31.3 wt. %, about 32.3 wt. %, about 33.3 wt. %, about 34.3 wt. %, about 35.3 wt. %, about 36.3 wt. %, about 37.3 wt. %, about 38.3 wt. %, about 39.3 wt. %, about 40.3 wt. %, about 41.3 wt. %, about 42.3 wt. %, about 43.3 wt. %, about 44.3 wt. %, about 45.3 wt. %, about 46.3 wt. %, about 47.3 wt. %, about 48.3 wt. %, about 49.3 wt. %, about 50.3 wt. %, about 51.3 wt. %, about 52.3 wt. %, or about 53.3 wt. %; wherein the wt. % is based on the total weight of the solid pharmaceutical compositions (powder blend or uncoated tablet).

In some embodiments, the solid pharmaceutical compositions (powder blend or uncoated tablet) of the disclosure comprises about 14.67 wt. % to about 28.0 wt. % of the SPD consisting essentially of milvexian free form and a pH-dependent enterosoluble polymer in a weight ratio (milvexian free form:pH-dependent enterosoluble polymer) of about 3:1, such as for example, about 14.67 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, or about 28.0 wt. %; wherein the wt. % is based on the total weight of the solid pharmaceutical compositions (powder blend or uncoated tablet).

In some embodiments, the solid pharmaceutical compositions (powder blend or uncoated tablet) of the disclosure comprises about 22.22 wt. % of the SPD consisting essentially of milvexian free form and a pH-dependent enterosoluble polymer in a weight ratio (milvexian free form:pH-dependent enterosoluble polymer) of about 3:1; wherein the wt. % is based on the total weight of the solid pharmaceutical compositions (powder blend or uncoated tablet).

In other embodiments, the present disclosure provides an immediate release, tablet core containing 25 mg or 100 mg milvexian free form having the following compositions as in Table 7 below.

TABLE 7
Immediate release tablet core (uncoated tablet)
of 25 mg and 100 mg strength for milvexian

| | Amount | | |
|---|---|---|---|
| | | Amount | Amount |
| | wt. | per dose | per dose |
| Component | % | (25 mg) | (100 mg) |
| spray-dried amorphous | 22.22 | 33.33 | 133.3 |
| solid dispersion of | | | |
| milvexian and HPMC- | | | |
| AS-MG in a weight-by- | | | |
| weight ratio of 3:1 | | | |
| Silicified Microcrystalline | 43.07 | 64.6 | 258.4 |
| cellulose | | | |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| Tablet Hardness | | preferred: 90N | preferred: 180N |
| | | Average: | Average: |
| | | 60N-120N | 140N-220N |
| | | (Ph. Eur. 2.9.8) | (Ph. Eur. 2.9.8) |
| Tablet friability | | <0.5% | <0.5% |
| Tablet disintegration | | 13 | 18 |
| time (seconds) | | | |

wherein, the immediate release tablet core for 25 mg or 100 mg strength of milvexian is further optionally coated with a film coating.

It has unexpectedly been found that the mixture of binder (e.g., SMCC 90) and Lactose Monohydrate in a 3:2 w/w weight ratio has desireable physical properties, including:
(i) free flow characteristics which allows mass transport during batch or continuous direct compression tableting process,
(ii) excellent compressibility which allows direct compression manufacture process to product tablet core with excellent physical stability such as friability less than 0.5%;
(iii) self lubricating properties with limited sticking to the wall of the tablet machine, and
(iv) sufficient rigidity to keep the shape of the SDP particles.

None of these advantageous properties has been reported in the prior art.

The specific excipients and their respective amounts used in the solid pharmaceutical composition for oral administration described herein were determined by screening excipients for their manufacturability in direct compression tableting methods and their impact on drug product quality, as measured by, for example, free flowing characteristics, compressibility in direct compression tableting processes, compatibility with milvexian, a 2-year shelf stability including physical, amorphous form, and chemical stability of milvexian, low friability (e.g., less than 1%), a hardness in a range from 60 N to 220 N, a disintegration time of less than 5 minutes. See Examples 1-4.

### Binder

In some aspects, the disclosure is directed to solid pharmaceutical compositions for oral administration comprising a binder.

In some embodiments of the solid pharmaceutical compositions for oral administration, the binder is selected from hydroxypropyl methylcellulose (HPMC), hydroxypropyl cellulose (HPC), microcrystalline cellulose (MCC), silicified microcrystalline cellulose (SMCC), polyvinylpyrrolidone (PVP), lactose, starch, or combination thereof.

In some embodiments of the solid pharmaceutical compositions for oral administration, the binder is selected from microcrystalline cellulose (MCC), silicified microcrystalline cellulose (SMCC), or combination thereof.

In some embodiments of the solid pharmaceutical compositions for oral administration, the binder consists of microcrystalline cellulose and silicified microcrystalline cellulose.

In some embodiments of the solid pharmaceutical compositions for oral administration, the binder is silicified microcrystalline cellulose (SMCC).

In some embodiments of the solid pharmaceutical composition comprises about 21.0 wt. % to about 71.0 wt. % binder, such as, for example, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. 0.0%, about 51.0 wt. %, about 52.0 wt. %, about 53.0 wt. %, about 54.0 wt. 0.0%, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. 0.0%, about 70.0 wt. %, or about 71.0 wt. % binder; wherein the wt. % is based on the total weight of the solid pharmaceutical composition.

In some embodiments of the solid pharmaceutical composition comprises about 21.0 wt. % to about 71.0 wt. % silicified microcrystalline cellulose (SMCC), such as, for example, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. 0.0%, about 51.0 wt. %, about 52.0 wt. %, about 53.0 wt. %, about 54.0 wt. 0.0%, about 56.0 wt. %, about 57.0 wt. %, about 58.0 wt. %, about 59.0 wt. %, about 60.0 wt. %, about 61.0 wt. %, about 62.0 w %, about 63.0 wt. %, about 64.0 wt. %, about 65.0 wt. %, about 66.0 wt. %, about 67.0 wt. %, about 68.0 wt. 0.0%, about 70.0 wt. %, or about 71.0 wt. % silicified microcrystalline cellulose (SMCC); wherein the wt. % is based on the total weight of the solid pharmaceutical composition.

In some embodiments of the solid pharmaceutical composition comprises about 31.0 wt. % to about 61.0 wt. % binder, such as, for example about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. 0.0%, about 51.0 wt. %, about 52.0 wt. %, about 53.0 wt. %, about 54.0 wt. 0.0%, about 56.0 wt. %, about 57.0 wt. %, about 58.0 wt. %, about 59.0 wt. %, about 60.0 wt. %, about 61.0 wt. % binder; wherein the wt. % is based on the total weight of the solid pharmaceutical composition.

In some embodiments of the solid pharmaceutical composition comprises about 31.0 wt. % to about 61.0 wt. % silicified microcrystalline cellulose (SMCC), such as, for example about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. 0, about 49.0 wt. 0.0%, about 51.0 wt. %, about 52.0 wt. %, about 53.0 wt. %, about 54.0 wt. 0.0%, about 56.0 wt. %, about 57.0 wt. %, about 58.0 wt. %, about 59.0 wt. %, about 60.0 wt. %, about 61.0 wt. % silicified microcrystalline cellulose (SMCC); wherein the wt. % is based on the total weight of the solid pharmaceutical composition.

In some embodiments of the solid pharmaceutical composition comprises about 38.0 wt. % to about 48.0 wt. % binder, such as, for example about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, binder; wherein the wt. % is based on the total weight of the solid pharmaceutical composition.

In some embodiments of the solid pharmaceutical composition comprises about 38.0 wt. % to about 48.0 wt. % silicified microcrystalline cellulose (SMCC), such as, for example about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, silicified microcrystalline cellulose (SMCC); wherein the wt. % is based on the total weight of the solid pharmaceutical composition.

In some embodiments, the solid pharmaceutical composition comprises about 25.0 wt. % to about 50.0 wt. % silicified microcrystalline cellulose, such as, for example, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, or about 50.0 wt. % silicified microcrystalline cellulose; wherein the wt. % is based on the total weight of the solid pharmaceutical composition.

In some embodiments, the solid pharmaceutical composition comprises about 35.0 wt. % to about 50 wt. % silicified microcrystalline cellulose, such as, for example, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, or about 50.0 wt. % silicified microcrystalline cellulose; wherein the wt. % is based on the total weight of the solid pharmaceutical composition.

In some embodiments, the solid pharmaceutical composition comprises about 40.0 to about 45.0 wt. % silicified microcrystalline cellulose, such as, for example, about 40.0 wt. %, about 41.0 wt. %, about 42.0 wt. %, about 43.0 wt. %, about 44.0 wt. %, or about 45.0 wt. % (wt/wt) silicified microcrystalline cellulose; wherein the wt. % is based on the total weight of the solid pharmaceutical composition.

In other embodiments, the solid pharmaceutical composition comprises about 40.0 wt. % to about 45.0 wt. % silicified microcrystalline cellulose based on the total weight of the solid pharmaceutical composition.

In some embodiments, the solid pharmaceutical composition comprises about 43.0 wt. % silicified microcrystalline cellulose based on the total weight of the solid pharmaceutical composition.

In some embodiments, the solid pharmaceutical composition comprises about 43.07 wt. % silicified microcrystalline cellulose based on the total weight of the solid pharmaceutical composition.

Microcrystalline cellulose is refined wood pulp. It is a white, free-flowing powder. Chemically, it is an inert substance, is not degraded during digestion and has no appreciable absorption. In large quantities it provides dietary bulk and may lead to a laxative effect. Tablets can be formed that are hard, but dissolve quickly. Microcrystalline cellulose is the same as cellulose, except that it meets USP standards. In some embodiments, for the solid oral dosage formulations described herein, the commercially available microcrystalline cellulose may comprise MCC sold under the trademark Avicel PH102® (Dupont Pharma).

Silicified microcrystalline cellulose functions as a filler for the solid oral dosage formulation described herein. It is a commercially available, intimate physical mixture of 2 components: microcrystalline cellulose (98% w/w) and colloidal silicon dioxide (2% w/w). At low magnification, traditional and silicified MCC look very similar in terms of their particle size and shape. At high magnification, however, electron microscopy reveals the differentiation in the microstructures of silicified MCC and traditional MCC. There are a wide variety grades of PROSOLV SMCC® with different physical properties (see Table 8 below).

TABLE 8
SMCC grades

| | Average Particle Size by | Bulk Density |
|---|---|---|
| Grade | Laser Diffraction (μm) | (g/mL) |
| PROSOLV ® SMCC 50 | 65 | 0.25-0.37 |
| PROSOLV ® SMCC 50 LD | 50 | 0.20-0.30 |
| PROSOLV ® SMCC 90 | 125 | 0.25-0.37 |
| PROSOLV SMCC HD 90 | 125 (90-160) | 0.38-0.50 |
| PROSOLV SMCC 90 LM | 125 | 0.27-0.39 |

In some embodiments of the solid pharmaceutical compositions described herein, the SMCC has the trademark PROSOLV SMCC® and is a grade selected from PROSOLV SMCC®50, PROSOLV SMCC® 50 LD, PROSOLV SMCC® 90, PROSOLV SMCC® HD 90, or PROSOLV SMCC® 90 LM grades as described in Table 8 above.

In a most preferred embodiment of the solid pharmaceutical compositions described herein, the SMCC is presented in an amount at about 43.0 wt. % based on the total weight of the solid pharmaceutical formulation. It has been found that this amount of binder allows production of a direct compressed tablet having favorable properties, e.g., a friability at less than 0.5%, and a disintegration time less than 2 minutes, and in many cases a disintegration time less than 30 seconds.

### Filler

In some aspects, the solid pharmaceutical compositions of the disclosure comprise a filler.

In some embodiments of the solid pharmaceutical compositions of the disclosure, the filler is selected from lactose, mannitol, or combination thereof.

In some embodiments of the solid pharmaceutical compositions of the disclosure, the filler is lactose.

In some embodiments, the lactose is lactose anhydrous, or lactose monohydrate.

In some embodiments, the lactose is lactose monohydrate.

In some embodiments, the filler consists of lactose monohydrate.

In some embodiments, the lactose monohydrate is that which is sold under the trade name Supertab 11SD® (DFE Pharma.).

In some embodiments of the solid pharmaceutical compositions described herein, the filler is present in the compositions in an amount of about 25.0 wt % to 33.0 wt % by weight, such as, for example, about 25.0 wt %, about 26.0 wt %, about 27.0 wt %, about 28.0 wt %, about 29.0 wt %, about 30.0 wt %, about 31.0 wt %, about 32.0 wt %, or about 33.0 wt %; wherein the wt. % is based on the total weight of the solid pharmaceutical composition.

In some embodiments of the solid pharmaceutical compositions described herein, the lactose monohydrate is present in the compositions in an amount of about 25.0 wt. % to 33.0 wt. %, such as, for example, about 25.0 wt. %, about 26.0 wt. %, about 27.0 wt. %, about 28.0 wt. %, about 29 wt. %, about 30 wt. %, about 31 wt. %, about 32 wt. %, or about 33 wt. % wherein the wt. % is based on the total weight of the solid pharmaceutical composition.

In some embodiments of the pharmaceutical compositions described herein, the filler is present in the compositions in an amount of about 28.0 wt. % to about 30.0 wt. %, such as, for example, about 28.0 wt. %, about 29.0 wt. %, or about 30.0 wt. %; wherein the wt. % is based on the total weight of the solid pharmaceutical composition.

In some embodiments of the pharmaceutical compositions described herein, the lactose monohydrate is present in the compositions in an amount of about 28.0 wt. % to about 30 wt. %, such as, for example, about 28.0 wt. %, about 29.0 wt. %, or about 30.0 wt. %; wherein the wt. % is based on the total weight of the solid pharmaceutical composition.

In some embodiments of the pharmaceutical compositions described herein, the filler is present in the compositions in an amount of about 29.0 wt. % based on the total weight of the solid pharmaceutical composition.

In some embodiments of the pharmaceutical compositions described herein, the lactose monohydrate is present in the compositions in an amount of about 29.0 wt. % based on the total weight of the solid pharmaceutical composition.

In some embodiments of the pharmaceutical compositions described herein, the filler is present in the compositions in an amount of about 28.0 wt. % based on the total weight of the solid pharmaceutical composition.

In some embodiments of the pharmaceutical compositions described herein, the lactose monohydrate is present in the compositions in an amount of about 28.0 wt. % based on the total weight of the solid pharmaceutical composition.

In some embodiments of the pharmaceutical compositions described herein, the filler is present in the compositions in an amount of 28.71 wt. % based on the total weight of the solid pharmaceutical composition.

In some embodiments of the pharmaceutical compositions described herein, the lactose monohydrate is present in the compositions in an amount of 28.71 wt. % based on the total weight of the solid pharmaceutical composition.

### Binder to Filler Weight Ratio

The weight ratio of binder to filler has been found to be important for imparting certain desirable characteristics to the solid pharmaceutical compositions for oral administration, including, for example friability and stickiness properties to the direct compressed tablets of the disclosure.

In some embodiments, the weight ratio of binder to filler ranges from about 100:0 to 0:100.

In some embodiments, the weight ratio of binder to filler ranges from about 99:1 to 0.5:1.

In preferred embodiments, the weight ratio of binder to filler ranges from about 3:2 to about 3:1, such as, for example, about 3:2, about 3:1.9, about 3:1.8, about 3:1.7, about 3:1.6, about 3:1.5, about 3:1.4, about 3:1.3, about 3:1.2, about 3:1.1, or about 3:1.

In preferred embodiments, the weight ratio of silicified microcrystalline cellulose (SMCC) to lactose monohydrate ranges from about 3:2 to about 3:1, such as, for example, about 3:2, about 3:1.9, about 3:1.8, about 3:1.7, about 3:1.6, about 3:1.5, about 3:1.4, about 3:1.3, about 3:1.2, about 3:1.1, or about 3:1.

In another preferred embodiments, the weight ratio of silicified microcrystalline cellulose (SMCC) to lactose monohydrate is about 3:2.

In preferred embodiments, the weight ratio of microcrystalline cellulose (MCC) to lactose monohydrate ranges from about 3:2 to about 3:1, such as, for example, about 3:2, about 3:1.9, about 3:1.8, about 3:1.7, about 3:1.6, about 3:1.5, about 3:1.4, about 3:1.3, about 3:1.2, about 3:1.1, or about 3:1.

In another preferred embodiments, the weight ratio of microcrystalline cellulose (MCC) to lactose monohydrate is about 3:2.

It has been found that this ratio of binder:lactose monohydrate of about 3:2 to about 3:1 imparts desired non-stickiness to the composition such that the composition does not stick to the walls of the tableting machine. This feature enables tablet production by continuous manufacturing processes, which, in turn, allow cost-effective commercial manufacturing. See Example 2A.

### Disintegrant

In some aspects, the solid pharmaceutical compositions of the disclosure comprise a disintegrant.

In some embodiments of the solid pharmaceutical compositions described herein, the disintegrant is selected from crosslinked sodium carboxymethyl cellulose (croscarmellose sodium, CCS), crosslinked polyvinylpyrrolidone (crospovidone, CPV), or combinations thereof.

In a preferred embodiment of the solid pharmaceutical compositions described herein, the disintegrant is croscarmellose sodium. Croscarmellose sodium is a cross-linked polymer of carboxymethylcellulose. In a preferred embodiment, the disintegrant is croscarmellose sodium sold under brand name Ac-di-sol SD-711 (Dupont Pharma. Delaware, US).

In some embodiments, the solid pharmaceutical compositions comprise disintegrant in an amount up to 10.0 wt. % disintegrant based on the total weight of the solid pharmaceutical composition, for example, up to 1.0 wt. %, up to 2.0 wt. %, up to 3.0 wt. %, up to 4.0 wt. %, up to 5.0 wt. %, up to 6.0 wt. up to 7.0 wt. %, up to 8.0 wt. %, up to 9.0 wt. %, or up to 10.0 wt. % of disintegrant.

In some embodiments, the solid pharmaceutical compositions comprise up to 10.0 wt. % croscarmellose sodium by the total weight of the solid pharmaceutical composition, for example, up to 1.0 wt. %, up to 2.0 wt. %, up to 3.0 wt. %, up to 4.0 wt. %, up to 5.0 wt. %, up to 6.0 wt. up to 7.0 wt. %, up to 8.0 wt. %, up to 9.0 wt. %, or up to 10.0 wt. % of croscarmellose sodium.

In some embodiments, the solid pharmaceutical compositions comprise about 2.0 wt. % to about 8.0 wt. % disintegrant by the total weight of the solid pharmaceutical composition, for example, about 2.0 wt. %, about 3.0 wt. %, about 4.0 wt. %, about 5.0 wt. %, about 6.0 wt. %, about 7.0 wt. %, or about 8.0 wt. % of disintegrant.

In some embodiments, the solid pharmaceutical compositions comprise about 2.0 wt. % to about 8.0 wt. % croscarmellose sodium by the total weight of the solid pharmaceutical composition, for example, about 2.0 wt. %, about 3.0 wt. %, about 4.0 wt. %, about 5.0 wt. %, about 6.0 wt. %, about 7.0 wt. %, or about 8.0 wt. % of croscarmellose sodium.

In some embodiments, the solid pharmaceutical compositions comprise about 3.0 wt. % to about 7.0 wt. % disintegrant based on the total weight of the solid pharmaceutical composition, such as, for example, about 3.0 wt. %, about 4.0 wt. %, about 5.0 wt. %, about 6.0 wt. %, or about 7.0 wt. % of disintegrant.

In some embodiments, the solid pharmaceutical compositions comprise about 3.0 wt. % to about 7.0 wt. % croscarmellose sodium based on the total weight of the solid pharmaceutical composition, for example, about 3.0 wt. %, about 4.0 wt. %, about 5.0 wt. %, about 6.0 wt. %, or about 7.0 wt. % of croscarmellose sodium.

In some embodiments, the solid pharmaceutical compositions comprise about 4.0 wt. % to about 6.0 wt. % disintegrant based on the total weight of the solid pharmaceutical composition, for example, about 4.0 wt. %, about 5.0 wt. %, or about 6.0 wt. % disintegrant.

In some embodiments, the solid pharmaceutical compositions comprise about 4.0 wt. % to about 6.0 wt. % croscarmellose sodium based on the total weight of the solid pharmaceutical composition, for example, about 4.0 wt. %, about 5.0 wt. %, or about 6.0 wt. % croscarmellose sodium.

In some embodiments, the solid pharmaceutical compositions comprise about 5.0 wt. % disintegrant based on the total weight of the solid pharmaceutical composition.

In some embodiments, the solid pharmaceutical compositions comprise about 5.0 wt. % croscarmellose sodium based on the total weight of the solid pharmaceutical composition.

It has been found this amount of croscarmellose sodium results in pharmaceutical compositions that can form direct compressed tablets having a friability at less than 0.5%, a disintegration time less than 2 minutes. See Examples 2D and 3A-C.

### Lubricant

In some aspects, the solid pharmaceutical compositions of the disclosure comprise a lubricant.

In some embodiments of the solid pharmaceutical compositions of the disclosure, the lubricant is selected from vegetable stearin, magnesium stearate, stearic acid, or combinations thereof.

In some embodiments of the solid pharmaceutical compositions of the disclosure the lubricant is magnesium stearate.

In some embodiments of the solid pharmaceutical compositions of the disclosure the lubricant is vegetable sourced magnesium stearate.

In some embodiments, the solid pharmaceutical compositions of the disclosure comprise lubricant in an amount up to 3.0 wt. % based on the total weight of the solid pharmaceutical composition, for example, up to 1.0 wt. %, up to 2.0 wt. %, or up to 3.0 wt. % lubricant.

In some embodiments, the solid pharmaceutical compositions of the disclosure comprise up to 3.0 wt. % magnesium stearate based on the total weight of the solid pharmaceutical composition, for example, up to 1.0 wt. %, up to 2.0 wt. %, or up to 3.0 wt. % magnesium stearate.

In a preferred embodiment, the solid pharmaceutical compositions of the disclosure comprise about 0.5 wt. % to about 2.0 wt. % lubricant based on the total weight of the solid pharmaceutical composition, for example, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2.0 wt. % lubricant.

In a preferred embodiment, the solid pharmaceutical compositions of the disclosure comprise about 0.5 wt. % to about 2.0 wt. % magnesium stearate based on the total weight of the solid pharmaceutical composition, for example, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2.0 wt. % magnesium stearate.

In a most preferred embodiment, the solid pharmaceutical compositions of the disclosure comprise about 0.5 wt. % to about 1.5 wt. % lubricant based on the total weight of the solid pharmaceutical composition, for example, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, or about 1.5 wt. % lubricant.

In a most preferred embodiment, the solid pharmaceutical compositions of the disclosure comprise about 0.5 wt. % to about 1.5 wt. % magnesium stearate based on the total weight of the solid pharmaceutical composition, for example, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, or about 1.5 wt. % magnesium stearate.

In a most preferred embodiment, the solid pharmaceutical compositions of the disclosure comprise about 1.0 wt. % lubricant based on the total weight of the solid pharmaceutical composition.

In a most preferred embodiment, the solid pharmaceutical compositions of the disclosure comprise about 1.0 wt. % magnesium stearate based on the total weight of the solid pharmaceutical composition.

### Film-Coated Pharmaceutical Tablets

### Film Coating

In some aspects of the disclosure, the solid pharmaceutical composition of the disclosure is direct compressed into a tablet core, wherein the tablet core is then film-coated to produce a film coated tablet.

In some embodiments, the film coating comprises polyvinyl alcohol, titanium dioxide, polyethylene glycol-polyvinyl alcohol graft copolymer, and talc.

In other embodiments, the film coating comprises polyethylene glycol-polyvinyl alcohol graft copolymer.

In some embodiments, the film coating is selected from the group consisting of film coating comprising polyvinyl alcohol (PVA) and 20% polyethylene glycol (PEG); hydroxypropylmethylcellulose (HPMC); polyvinyl alcohol and polyethylene glycol graft polymer (film coating sold under trade name Opadry® QX Colorcon); and polyvinyl alcohol free of PEG (e.g., film coating sold under trade name Opadry® AMB II, Colorcon).

In some embodiments, the film coating is selected from the group consisting of PVA+PEG (Opadry II 85F220241), HPMC (Opadry II 32F220042). PVA (Opadry amb II 88A520052), and PVA/PEG (Opadry QX 321A220057).

In such embodiments, the Opadry® QX film coating comprises polyvinyl alcohol, titanium dioxide, macrogol (PEG) polyvinyl alcohol grafted copolymer, and talc. Opadry® QX grade film coating material, an example of such a coating, has the composition in Table 9 below. In some embodiments, the film coating is free of titanium dioxide.

TABLE 9
Opadry ® QX 321A220063 Yellow composition

Components
Macrogol (PEG) polyvinyl alcohol grafted copolymer
talc
Titanium dioxide
Glycerol monocapry locaprate Type I
Polyvinyl alcohol
Iron oxide yellow

In some embodiments of the film-coated tablets of the disclosure, the film coating comprises about 2.0% to about 4.0% of weight-gain by the uncoated tablet, for example, about 2.0% about 2.1% about 2.20% about 2.3%, about 2.4% about 2.5%, about 2.6% about 2.7% about 2.8% about 2.9% about 3.0% about 3.1% about 3.2%, about 3.3%, about 3.4%, about 3.5%, about 3.6% about 3.7%, about 3.8% about 3.9%, or about 4.0%.

As used herein to describe the weight of coating in the film-coated tablets of the disclosure, the phrase “% of weight-gain by the uncoated tablet” refers to percentage of the weight of the uncoated tablet that is represented by the coating. This percentage is calculated as ([(weight of the coated tablet)−(weight of uncoated tablet)]÷(weight of uncoated tablet))*100. For example, if the coated tablet weighs 154.5 mg and the uncoated tablet weighs 150 mg, then the “% of weight-gain by the uncoated tablet equals [(154.5 mg−150 mg)÷150 mg]*100=3.0%.

In some embodiments of the film-coated tablets of the disclosure, the film coating comprises about 2.5% to about 3.3% of weight gain by the uncoated tablet, for example, about 2.5%, about 2.6%, about 2.7%, about 2.8%, about 2.9%, about 3.0%, about 3.1%, about 3.2%, or about 3.3%.

In some embodiments of the film-coated tablets of the disclosure, the film coating is about 3.0% of weight gain by the uncoated tablet.

The film coating may be applied by any coating method known to one skilled in the art.

In some embodiments, the present disclosure is directed to immediate release, film-coated tablet for 25 mg or 100 mg strength milvexian having the following compositions as in Table 10 below.

TABLE 10
Film coated Tablet of 25 mg and 100 mg strength

| | Amount | | |
|---|---|---|---|
| | | Amount per | Amount per |
| | | dose (25 mg) | dose (100 mg) |
| Component | wt. % | unit (mg) | unit (mg) |
| spray-dried amorphous solid | 22.22 | 33.33 | 133.3 |
| dispersion of milvexian and | | | |
| HPMC-AS-MG in a weight-by- | | | |
| weight ratio of 3:1 | | | |
| Silicified Microcrystalline | 43.07 | 64.6 | 258.4 |
| cellulose | | | |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 | | | |
| Yellow (mg) | 3.0 | 4.5 | 18.0 |
| Total weight (mg) | | 154.5 | 618 |

In an embodiment, this disclosure also provides a tablet for fast dispersion in aqueous media.

In some embodiments, the tablet for fast dispersion in aqueous media is characterized by having a disintegration time at less than 1 minutes and a hardness ranging from 60-200 N and comprising 25 mg or 100 mg of milvexian free form.

In some embodiments, the tablet for fast dispersion in aqueous media is characterized by having a disintegration time at less than 1 minutes and a hardness ranging from 60-120 N and comprising 25 mg of milvexian free form.

In some embodiments, the tablet for fast dispersion in aqueous media is characterized by having a disintegration time at less than 1 minutes and a hardness of 90 N and comprising 25 mg of milvexian free form.

In some embodiments, the tablet for fast dispersion in aqueous media is characterized by having a disintegration time at less than 1 minutes and a hardness ranging from 140-220 N and comprising 100 mg of milvexian free form.

In some embodiments, the tablet for fast dispersion in aqueous media is characterized by having a disintegration time at less than 1 minutes and a hardness of 180 N and comprising 100 mg of milvexian free form.

In some embodiments, the tablet for fast dispersion in aqueous media has the compositions described in Tables 5-7 and 10 above.

In some embodiments, the tablet for fast dispersion in aqueous media has the compositions described in Table 7 above. In some embodiments, the tablet for fast dispersion in aqueous media has the compositions described in Table 10 above.

In some embodiments, the disclosure provides an dispersion of amorphous solid dispersion (ASD) in an aqueous media selected from water, deionized water, saline, phosphate buffer, or fruit juice such as apple sauce, cranberry juice, orange juice, vegetable juice. In some embodiments, the aqueous dispersion of ASD may be administered to a patient who are unable to swallow medication via a feeding tube (e.g., NG tube) or a spoon.

In some aspects, the disclosure is directed to pharmaceutical tablets which are formed by a process comprising direct compression of the solid pharmaceutical compositions of the disclosure.

In some embodiments, the disclosure is directed to pharmaceutical tablets which are formed by a process comprising direct compression of the solid pharmaceutical compositions of the disclosure, wherein the process further comprises coating the tablet core formed by the direct compression process with a film coating.

In some embodiments, the film coating comprises polyvinyl alcohol, titanium dioxide, polyethylene glycol-polyvinyl alcohol graft copolymer, and talc: (e.g., Opadry®, QX grade sold by Colorcon).

In other embodiments, the film coating comprises polyethylene glycol-polyvinyl alcohol graft copolymer.

In some embodiments, the disclosure is directed to a pharmaceutical tablet comprising:
a. a core comprising:
   i. a spray-dried amorphous solid dispersion (SDP) consisting essentially of milvexian free form and a pH-dependent enterosoluble polymer;
   ii. a binder selected from microcrystalline cellulose (MCC), silicified microcrystalline cellulose (SMCC), or a combination thereof;
   iii. lactose monohydrate;
   iv. a disintegrant;
   v. a lubricant; and
b. a film coating covering the core;
wherein milvexian is present in an amount ranging from about 10.0 wt. % to about 40.0 wt. % of the total weight of the core; and
wherein the binder and lactose monohydrate are present in the core in a weight ratio (binder:lactose monohydrate) ranging from about 3:2 to about 3:1.

In these embodiments, the tablet core is a pharmaceutical composition of the disclosure, and the spray-dried amorphous solid dispersion (SDP), the binder selected from microcrystalline cellulose (MCC), silicified microcrystalline cellulose (SMCC), or a combination thereof; the lactose monohydrate, the disintegrant, and the lubricant are as those disclosed above with respect to the pharmaceutical compositions of the disclosure.

In some embodiments, the pharmaceutical tablets of the disclosure comprise about 25 mg of milvexian free form.

In other embodiments, the pharmaceutical tablets of the disclosure comprise comprises about 100 mg of milvexian free form.

In some embodiments, the pharmaceutical tablets of the disclosure have a friability of less than 0.5%.

In some embodiments, the pharmaceutical tablets of the disclosure have a disintegration time of less than 2 minutes.

In some embodiments, the pharmaceutical tablets of the disclosure have a disintegration time of less than 20 minutes.

In some embodiments, the pharmaceutical tablets of the disclosure have a disintegration time of less than 20 seconds.

In some embodiments, the core of the tablets of the disclosure are formed by direct compression of the solid pharmaceutical compositions of the disclosure.

Thus, in some embodiments, the tablet cores of the disclosure have the same weight percentage amount of milvexian, silicified microcrystalline cellulose, lactose monohydrate, disintegrant, and lubricant as set forth above with respect to the pharmaceutical powder blend.

In some embodiments, tablets of the disclosure comprise about 25 mg of milvexian free form.

In some embodiments, tablets of the disclosure comprise about 100 mg of milvexian free form.

In some aspects, the tablet of the disclosure has a hardness of about 50 N to about 140 N, about 60 N to about 120 N. In some aspects, the tablet of the disclosure has a hardness ranging from about 50 N to about 140 N. In some aspects, the tablet of the disclosure has a hardness ranging from about 60 N to about 120 N, for example, about 60 N, about 65 N, about 70 N, about 75 N, about 80 N, about 85 N, about 90 N, about 95 N, about 100 N, about 105 N, about 110 N, about 115 N, or about 120 N.

In other aspects, the tablet of the disclosure has a hardness of about 140 N to about 220 N, or about 100 N to about 260 N. In some aspects, the tablets of the disclosure has a hardness ranging from about 100 N to about 260 N. In a preferred embodiment, the tablet of the disclosure has a hardness ranging from about 140 N to about 220 N, for example, about 140 N, about 145 N, about 150 N, about 155 N, about 160 N, about 165 N, about 170 N, about 175 N, about 180 N, about 185 N, about 190 N, about 195 N, about 200 N, about 205 N, about 210 N, about 215 N, about 220 N.

In other aspects, the tablets of the disclosure has a friability of less than 1%, such as, for example, less than 0.9%, less than 0.8%, less than 0.7%, less than 0.6%, less than 0.5%, less than 0.4%, less than 0.3%, less than 0.2%, or less than 0.1%.

In some aspects, the tablets of the disclosure have a disintegration time of 5 minutes or less, such as for example, 5 minutes or less, 4.5 minutes or less, 4.0 minutes or less, 3.5 minutes or less, 3.0 minutes or less, 2.5 minutes or less, 2.0 minutes or less, 2.0 minutes or less, 1.5 minutes or less, 1.0 minutes or less, or 0.5 minutes or less.

In some aspects, the tablets of the disclosure have specific performance characteristics.

### Medicaments and Use

In some aspects, the disclosure is directed to methods of administering milvexian to a patient in need thereof, comprising orally administering to the patient a pharmaceutical composition of the disclosure.

In some aspects, the disclosure is directed to methods of administering milvexian to a patient in need thereof, comprising orally administering to the patient a tablet of the disclosure.

In some aspects, the disclosure is directed to methods of administering milvexian to a patient in need thereof, comprising dispersing the tablet of the disclosure in an aqueous medium and then administering the resulting dispersion to the patient through a feeding tube.

In some aspects, the disclosure is directed to methods of administering milvexian to a patient in need thereof, comprising dispersing the tablet of the disclosure in an aqueous medium, adding applesauce to the dispersion and mixing, and then administering the resulting mixture to the patient by mouth.

In some embodiments, the aqueous medium is water.

In some embodiments, the aqueous medium comprises a fruit juice or a vegetable juice, such as apple juice, orange juice, or cranberry juice.

In some aspects, the disclosure is directed to methods for treatment of, and/or prophylaxis of, a thromboembolic disorder in a patient in need thereof, comprising administering to the patient a tablet of the disclosure.

In other aspects, the disclosure is directed to methods of treatment of and/or prophylaxis of a thromboembolic disorder in a patient in need thereof, comprising dispersing a tablet of the disclosure in an aqueous medium, and then administering the resulting dispersion to the patient through a feeding tube.

In other aspects, the disclosure is directed to methods of treatment of and/or prophylaxis of a thromboembolic disorder in a patient in need thereof, comprising dispersing a tablet of the disclosure in an aqueous medium, adding applesauce to the dispersion and mixing, and then administering the resulting mixture to the patient by mouth.

In some embodiments, the thromboembolic disorder is unstable angina, an acute coronary syndrome, atrial fibrillation, myocardial infarction, cerebrovascular ischemic attacks, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from medical implants, devices, or procedures in which blood is exposed to an artificial surface that promotes thrombosis.

### EXAMPLES

The features of the instant invention involve the improved solvent based spray drying process for manufacture of spray dried SDP particles with particle size amenable for direct compression tableting process, e.g., spray dried SDP has a median particle size distribution D₅₀ ranging from 30 μm to 60 μm and film-coated direct compression tablet thereof. The invention is further exemplified and disclosed by the following non-limiting examples.

### ABBREVIATION

API: active pharmaceutical ingredient
ASD: amorphous solid dispersion
DCM: dichloromethane
DC tablet: direct compression tablet
RC tablet: roller compaction tablet
FFC: flow function coefficient
LOD: loss on drying
HPMC-AS MG: hydroxypropyl methylcellulose acetate succinate sold under trademark
AQOAT® AS-MG (Shin-Etsu Chemical Co., Ltd. (Niigata, Japan)).
Macrogol: polyethylene glycol
MeOH: methanol
PXRD: powder X-ray diffraction
SDP: spray-dried amorphous solid dispersion
rBA: relative bioavailability
rh: relative humidity
UHPLC: Ultra-High-Performance Liquid Chromatography
UV: ultraviolet

### ANALYTICAL METHODS

### 1. Particle Size Characterization.

Average particle size can be determined by Malvern light scattering, a laser scattering technique. In the example below, the particle size for spray dried ASD of milvexian/HPMC-AS-MG 3:1 (w/w) was measured use a Malvern particle size analyzer. Upon measurement completion, the sample cell was emptied and cleaned, refilled with suspension medium, and the sampling procedure repeated for a total of three measures.

### 2. Bulk and Tap Density Measurement.

Determination of the bulk density is done by weighing of a specified sample amount (50 g) with a precision of 0.1 g. The sample is poured into a 250 mL measuring cylinder, and after carefully levelling the surface of the powder bed the volume is read. The bulk density is then calculated as the sample weight divided by the read powder bed volume.

The tap density is measured by taking the bulk density sample still in the 250 mL measuring cylinder, placing and securing it on a shaker. The shaker is then set for 500 shaking movement and started. After 500 shaking movements the shaking is stopped and the powder bed volume is read. The tap density is then calculated as the sample weight divided by the read powder bed volume.

### 3. Tablet Friability Measurement.

Friability is the tendency of tablets to powder, chip, or fragment.

Friability on core tablets was determined according to the guideline described in USP <1216>. Friability was measured on a sample of whole tablets corresponding to 6.5 g. The tablets were carefully dedusted prior to testing. The tablets were accurately weighed and placed in the drum which was rotated for 100 times. The tablets were removed from the drum and accurately weighed after any dust was removed. A maximum mean weight loss from the samples of not more than 1.0% was considered acceptable. If obviously cracked, cleaved, or broken tablets were present the test would fail.

### 4. Tablet Hardness Measurement.

Tablet hardness (or crushing strength) is the load required to crush the tablet when placed on its edge cf USP <1217> Tablet Breaking Force.

Tablet thickness, hardness and diameter were determined using a diametral hardness tester (Kraemer Universal Test System UTS4.1).

### 5. Xrpd Method.

Powder x-ray diffraction (PXRD) data were recorded on a PANalytical XPertPRO or Empyrean diffractometer using monochronomatized Cu-K alpha 1 radiation, a position sensitive detector, at generator setting of 45 kV and 40 mA. The samples were collected in transition or reflection mode. The scanning range was between 3° and 50° theta with a 0.1° or 0.2° step at minimally 60 seconds/step.

### 6. Disintegration Time Method.

Disintegration time is the time required for the tablet to break into particles under a given set of conditions.

The disintegration time was determined (n=6) using the apparatus described in Eur. Ph. (PTZ-E Pharma Test, Hamburg, Germany). Tests were performed in distilled water at 37° C. using disks.

### 7. Powder Flowability Tests.

Powder flowability can be measured using several methods and types of equipment. One commonly used method for pharmaceutical powders is “Shear Testing” measuring powder behavior as it transitions from no-flow to flow. The testing can be described as follows (see https://www.freemantech.co.uk/powder-testing/ft4-powder-rheometer-powder-flow-tester/shear-testing):

At very low speeds, a shear (or horizontal) force is applied to an upper layer of powder whilst the adjacent lower layer is prevented from moving (or vice versa). The force continues to increase but no relative movement at the shear plane occurs until the shear force is sufficiently high to overcome the powder's shear strength, at which point the powder bed ‘yields’ and the upper layer of powder slips against the lower.

In a typical shear cell test sequence, several shear tests would be carried out at different levels of normal stress. The data produced represents the relationship between shear stress and normal stress, which can be plotted to define the powder's Yield Locus.

It is possible to apply a number of mathematical models to this data, but it is important to consider that in doing so. trends may be exaggerated or reduced. Fitting Mohr stress circles to the yield locus identifies the Major Principal Stress (Sigma 1) and Unconfined Yield Strength (Sigma c), and the ratio of the former to the latter quantifies the Flow Function. FF. Flow Function is a parameter commonly used to rank flowability, with values below 4 denoting poor flow and above 10, good flow.

### Example 1. Preparation of Amorphous Solid Dispersion of Milvexian in HPMC-AS-MG

For all the spray drying processes described in the Examples 1b-1g below, the optimization of the spray drying process was first performed on medium scale spray dryer with post drying carried out in a vacuum drying oven. Later, it was transferred to commercial scale spray dryer with post drying carried out using a dynamic post dryer. It was found that the manufacture of the ASD containing 750 mg of milvexian and 250 mg HPMC-AS-MG on both medium scale and commercial scalespray dryer resulted in powders with similar physical properties, that is, large particle size, high bulk/tap density and excellent flow properties The manufactured powders manufactured on the medium and commercial exhibited similar physical properties right after the spray drying (termed wet spray dried powder). However, upon post drying on commercial scale a surprising increase in bulk/tap density was seen compared to the powder manufactured on medium scale due to the use dynamic drying instead of tray drying and taking advantage of the brittleness of the spray dried powder caused by the high amount of milvexian in the amorphous solid dispersion.

### Example 1a. Solubility of Milvexian in Various Organic Solvents

A main challenge in preparing a spray dried powder for direct compression is to obtain a spray dried powder that has a suitable flowability, large particle size and high density. A key here is to obtain the highest possible dissolved solids concentration in a given organic solvent mixture. Preferably the solvent mixture also has a low boiling point to facilitate fast evaporation and keep the drying temperature low (in case of issues regarding low glass transition temperature) and short drying times for large droplets/particles. The dissolved solids content and formulation should result in a viscosity high enough to create large droplets, while still being pumpable. The high dissolved solids content in a droplet for spray drying generally will result in higher density particles, and also give possibilities to engineer the particles towards better downstream manufacturability.

A previous spray drying solvent mixture for milvexian was the Acetone/Water mixtures disclosed in WO2020212629, with the preferred one being Acetone/Water 90:10 w/w ratio with a milvexian solubility of 36 mg/mL at 20° C. and 70 mg/mL at 50° C. resulting in solids content for spray drying of 5 wt. % and 8 wt. %, respectively. While heating of the feed solution is a viable solution for production, it is a more complex set-up and with risk of crashing out dissolved solids in case of cold spots and plant safety. Various solvent systems (See Table 11 below) were investigated with primary focus on solvent systems containing DCM due to its low boiling point and excellent solubilization properties.

TABLE 11
Dissolution Property of Milvexian in Different Solvent System

| | | Solubility | |
|---|---|---|---|
| | | in mg/mL | Solubility |
| Solvent | Temperature | solution | description |
| 100% DCM | 20 | 29 | Sparingly soluble |
| 100% DCM | 40 | 51 | Soluble |
| 100% MeOH | 20 | 4.2 | Slightly soluble |
| 100% MeOH | 40 | 6.6 | Slightly soluble |
| 30/70 DCM/MeOH | 20 | 16 | Sparingly soluble |
| 30/70 DCM/MeOH | 40 | 26 | Sparingly soluble |
| 40/60 DCM/MeOH | 20 | 29 | Sparingly soluble |
| 40/60 DCM/MeOH | 40 | 44 | Soluble |
| 50/50 DCM/MeOH | 20 | 58 | Soluble |
| 50/50 DCM/MeOH | 40 | 77 | Soluble |
| 60/40 DCM/MeOH | 20 | 120 | Freely Soluble |
| 60/40 DCM/MeOH | 40 | >200 | Freely Soluble |
| 70/30 DCM/MeOH | 20 | >200 | Freely Soluble |
| 70/30 DCM/MeOH | 40 | >200 | Freely Soluble |
| 80/20 DCM/MeOH | 20 | >200 | Freely Soluble |
| 80/20 DCM/MeOH | 40 | >200 | Freely Soluble |
| 90/10 DCM/MeOH | 20 | >200 | Freely Soluble |
| 90/10 DCM/MeOH | 40 | >200 | Freely Soluble |

The results in the table show that highly increased solubility is obtained when using DCM/MeOH systems at 20° C. of more than 200 mg/mL and thereby eliminating the need to heat the solution to increase solubility. Based on the data it was determined that 70/30 w/w % DCM/MeOH and 80/20 w/w % were promising solvent mixtures and more accurate solubility determination was carried out for 80/20 w/w % DCM/MeOH for both FORM A and P1.Acetone solvate at 20° C. with the results shown in Table 12 below.

TABLE 12
Solubility of P1.acetone in Different Solvent System

| | | Solubility | Solubility |
|---|---|---|---|
| | | in mg/mL | in mg/mL |
| | | solution | solution |
| Solvent | Milvexian form | 24 hours | 2 weeks |
| 70/30 DCM/MeOH | FORM A | 221 | 204 |
| 80/20 DCM/MeOH | FORM A | 272 | 255 |
| 100% MeOH | P1.Acetone solvate | 4.6 | 4.8 |
| 60/40 DCM/MeOH | P1.Acetone solvate | 102 | 99 |
| 70/30 DCM/MeOH | P1.Acetone solvate | — | 160 |
| 80/20 DCM/MeOH | P1.Acetone solvate | 226 | 227 |
| 90/10 DCM/MeOH | P1.Acetone solvate | 222 | 225 |
| 95/5 DCM/MeOH | P1.Acetone solvate | 163 | 162 |
| 100% DCM | P1.Acetone solvate | 33 | 33 |

The result show solubilities of >220 mg/mL was achievable for both milvexian Form A, and P1.Acetone milvexian forms. P1.Acetone was the preferred crystalline form of milvexian for physical stability and manufacturability reasons. Based on the above, 80/20 w/w % DCM/MeOH solvent system was selected for development due to highly increase solubility over the 90/10 w/w % Acetone/Water solvent system.

### Example 1b. SDP Prepared Using 70/30 w/w % DCM/MeOH

In initial testing, spray drying of about 11.25 wt. % of milvexian FORM A with 3.75 wt. % HPMC-AS MG in a solvent mixture containing 70/30 w/w % DCM/MeOH was carried out. Milvexian FORM A started dissolving immediately giving a clear yellow solution. After 10 min stirring at 21° C., about 3.75 wt. % of HPMC-AS MG (AQOAT® AS-MG, Shin-Etsu Chemical Co., Ltd. (Niigata, Japan)) was added to solution of milvexian. Stirring was continued for 5 min resulting in a slightly turbid yellowish solution of milvexian and HPMC-AS-MG. The viscosity of this turbid yellowish solution was 21 mPa·s at 20° C. The turbid yellowish solution was spray dried using a Buchi B-290 spray dryer with about 35 Kg/hr drying gas flow-rate capacity and equipped with a 2-fluid nozzle using the following parameters: Atomization gas flow rate set at 25 mm (301 L/hr); feed rate at 7.7 g/min, inlet/outlet temperatures at 70/43° C., condenser temperature of −20° C., spray nozzle orifice diameter of 0.7 mm and spray nozzle cap diameter of 1.4 mm. The spray drying process went on for 11 min to give 15.30 g of wet ASD (98% yield). The wet ASD was then subjected to drying for 24 hours in a vacuum oven (Heraeus, Model VT6130 M) at 40° C., with nitrogen flow, and a vacuum of approximately 250 mbar to give 14.40 g (92% yield) of desired SDP product. The SDP product was a white powder having an assay value of 99.5% and a purity of 100.0% by HPLC. The PXRD diffraction pattern showed a halo pattern with no crystalline peaks indicating the product is amorphous.

### Example 1c. SDP Prepared Using 70/30 w/w % DCM/MeOH

A solution containing about 12.3 wt. % of milvexian P1.Acetone (equivalent to about 11.25 wt. % of milvexian free form) and 3.75 wt. % HPMC-AS MG was prepared in a solvent mixture of 70/30 w/w % DCM/MeOH. Milvexian P1.Acetone started dissolving immediately, but the solution remained slightly turbid. After 10 min stirring at room temperature (i.e., 21° C.), about 250 mg (3.75 wt. %) of HPMC-AS MG (AQOAT® AS-MG, Shin-Etsu Chemical Co., Ltd. (Niigata, Japan)) was added to solution of milvexian. Stirring was continued for 5 min resulting in a turbid yellowish solution of milvexian and HPMC-AS-MG. The viscosity of this turbid yellowish solution was 17 mPa·s at 20° C. The turbid yellowish solution was spray dried using a Buchi B-290 spray dryer with about 35 Kg/hr drying gas flow-rate capacity and equipped with a 2-fluid nozzle using the following parameters: Atomization gas flow rate set at 25 mm (301 L/hr); feed rate at 7.8 g/min, inlet/outlet temperatures at 67/46° C., condenser temperature of −20° C., spray nozzle orifice diameter of 0.7 mm and spray nozzle cap diameter of 1.4 mm. The spray drying process went on for 11 min to give 12.08 g of wet ASD (93% yield). The wet ASD was then subjected to drying for 24 hours in a vacuum oven (Heraeus, Model VT6130 M) at 40° C., with nitrogen flow, and a vacuum of approximately 250 mbar to give 11.33 g (87% yield) of desired SDP product. The SDP product was a white powder having an assay value of 101.4% and a purity of 99.8% by HPLC. The PXRD diffraction pattern showed a halo pattern with no crystalline peaks indicating the product is amorphous.

Due to the turbid appearance of the solution when dissolving the milvexian (P1.Acetone) in 70/30 w/w % DCM/MeOH, a solubility test was performed by adding 3.75 g of milvexian (P1.Acetone) to a mixture of 19.83 g DCM and 8.5 g MeOH (DCM/MeOH 70/30 w/w %) under stirring by a magnetic stirrer. The appearance of the solution was evaluated at different timepoints over 20 hours. The solution was mixed for approximately 20 hours, but it still remained turbid, leading to the conclusion that the milvexian was not fully dissolved. No crystalline material was present, however, suggesting that the amount of undissolved material was small.

The SDPs from Examples 1b and 1c were stored at different storage conditions, as presented in the table below, to evaluate their physical and chemical stability. SEM analysis showed for both SDPs and at all storage conditions spherical and ‘imploded’ spherical particles indicating an amorphous solid state. For the SDP of Example 1a this was also supported by synchroton-XRD analysis where no traces of crystalline drug substance were found. PXRD analysis showed that both ASDs were amorphous at all storage conditions tested (See Table 13 below). Modulated DSC analysis of both ASDs showed a stable glass transition temperature (Tg) dependence on time in the range of 144-147° C.

TABLE 13
Assay and Purity of ASDs Example 1b and Example 1c

| | | | Example 1b | Example 1c | | |
|---|---|---|---|---|---|---|
| Storage | | | Assay of | Assay of | RRT0.61 | RRT 0.83 |
| conditions | Time | | Milvexian (%) | Milvexian (%) | (%) | (%) |
| 25° C./60% RH | Initial | | 99.5 | 101.4 | 0.06 | 0.05 |
| 50° C./75% RH | 1 | month | 98.0 | 97.2 | 0.05 | <0.05 |
| 60° C./75% RH | 1 | month | 100.0 | 96.9 | 0.06 | <0.05 |
| 70° C./10% RH | 14 | days | 99.9 | 95.9 | 0.05 | <0.05 |
| | 1 | month | 100.7 | 98.3 | 0.05 | 0.05 |
| 70° C./75% RH | 14 | days | 99.6 | 95.0 | 0.05 | <0.05 |
| | 1 | month | 99.6 | 95.9 | 0.06 | <0.05 |

### Example 1d. SDP Prepared Using 80/20 w/w % DCM/MeOH

A solution containing about 12.3 wt. % of milvexian P1.Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture of 80/20 w/w % DCM/MeOH was prepared. The P1.acetone and HPMC-AS MG dissolved immediately resulting in a light yellow clear solution. The clear solution was then spray dried at room temperature (i.e., 21° C.) using a Buchi B-290 spray dryer with a 35 Kg/hr drying gas flow-rate capacity, set of the following parameters: Atomization gas flow rate of 25 mm (301 L/hr); feed rate at 7.7 g/min; inlet/outlet temperature of 64/45° C., condenser temperature −20° C., spray nozzle orifice diameter of 0.7 mm and spray nozzle cap diameter of 1.4 mm. The spray drying process went on for 11 min to give 9.9 g (76% yield) of wet SDP. The wet SDP was subject to drying for 24 hours in a vacuum oven (Heraeus, Model VT6130 M) at 40° C., with nitrogen flow, and a vacuum of approximately 250 mbar to give 9.3 g (72% yield) of desired dry SDP.

The SDP product was a white powder having an assay of 97.7% and a purity of 99.9 by HPLC. The PXRD diffraction pattern showed a halo pattern with no crystalline peaks indicating the product is amorphous.

The resulting spray dried SDP was evaluated for manufacturability and tested for assay and impurities, residual solvents, and solid state. Assay was 97.7% and purity was 99.9%. Residual solvent levels after spray drying were: Methanol <50 ppm, Acetone 278 ppm, and Methylene chloride 118 ppm. All levels are well below the required solvent levels as specified by ICH Q3C Guideline. Overall, the results demonstrate the formulation has good manufacturability. Solid state NMR analysis showed the powder to be amorphous and having a glass transition temperature of about 145°.

### Example 1e. SDP Prepared Using 80/20 w/w % DCM/MeOH

A solution containing about 12.3 wt. % of milvexian P1.Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture containing 80/20 w/w % DCM/MeOH was prepared. The milvexian P1.Acetone and HPMC-AS MG dissolved immediately resulting in a light yellow clear solution. The clear solution was spray dried at room temperature (i.e., 21° C.) using a Buchi B-290 spray dryer with a 35 kg/hr drying gas flow-rate capacity, set of the following parameters: Atomization gas flow rate at 25 mm (301 L/h); feed rate at 7.5 g/min; inlet/outlet temperature at 65/43° C., condenser temperature −20° C., spray nozzleorifice diameter of 0.7 mm and spray nozzle cap diameter of 1.4 mm. The spray drying process went on for 11 min to give 10.4 g (82% yield) of wet SDP. The wet SDP was subject to drying for 24 hours in a vacuum oven (Heraeus, Model VT6130 M) at 40° C., with nitrogen flow, and vacuum of approximately 200 mbar to give 9.6 g (76% yield) of desired dry SDP.

The SDP product was a white powder having an assay of 96.7% and a purity of 99.9% by HPLC. The PXRD diffraction pattern showed a halo pattern with no crystalline peaks indicating the product is amorphous.

The resulting spray dried SDP was evaluated for manufacturability and tested for assay and impurities, residual solvents, and solid state. Assay was 96.7% and purity was 99.9%. Residual solvent levels after spray drying were: Methanol <50 ppm, Acetone 310 ppm, and Methylene chloride 117 ppm. All levels are well below the required solvent levels as specified by ICH Q3C Guideline. Overall, the results demonstrate the formulation has good manufacturability. Solid state NMR analysis showed the powder to be amorphous and mDSC analysis showed a glass transition temperature of about 144° C.

### Example 1f. SDP Prepared Using 80/20 w/w % DCM/MeOH

A solution containing about 12.3 wt. % of milvexian P1.Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture containing 80/20 w/w % DCM/MeOH was prepared. The milvexian P1.acetone and HPMC-AS MG dissolved quite immediately resulting in a light yellow clear solution. The clear solution at 21° C. was spray dried using a Buchi B-290 spray dryer with a 35 Kg/hr drying gas flow-rate capacity, set of the following parameters: Atomization gas flow rate at 25 mm (301 L/hr); feed rate at 7.7 g/min; inlet/outlet temperatures at 67/44° C., condenser temperature of −19° C., spray nozzle orifice diameter of 0.7 mm, and spray nozzle cap diameter of 1.4 mm. The spray drying process went on for 11 min to give 11.5 g (89% yield) of wet SDP. The wet SDP was subject to drying for 24 hours in a vacuum oven (Heraeus, Model VT6130 M) at 40° C., with nitrogen flow, and a vacuum of approximately 200 mbar to give 10.7 g (83% yield) of desired dry SDP.

The SDP product was a white powder having an assay of 98.8% and a purity of 99.9% by HPLC. The PXRD diffraction pattern showed a halo pattern with no crystalline peaks indicating the product is amorphous.

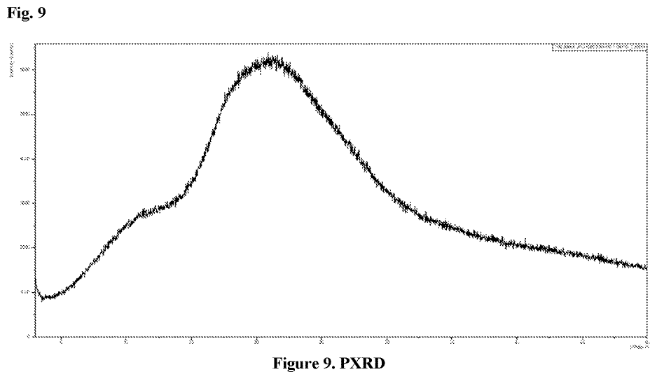

The resulted spray dried SDP were evaluated towards manufacturability and tested for assay and impurities, residual solvents, and solid state. Assay was 98.8% and purity was 99.9%. Residual solvent levels after spray drying were: Methanol <50 ppm, Acetone 225 ppm, and Methylene chloride <52 ppm. All levels are well below the required solvent levels as specified by ICH Q3C Guideline. Overall the results demonstrate the formulation has good manufacturability. Solid state NMR analysis showed the powder to be amorphous and mDSC analysis showed a glass transition temperature of about 144° C. PXRD diffraction pattern showed a halo pattern with no crystalline peaks indicating the product is amorphous (FIG. 9).

Based on the result of the incomplete solubility (turbidity remained after 20 hours) of milvexian in the solvent system comprising DCM/MeOH 70/30 w/w % as in Example 1c, and the clear feed solutions of Examples 1d-1f above, and the comparable stability of spray dried powders manufactured from milvexian P1.Acetone form relative to milvexian crystalline Form A form as in Example 1b, the volume ratio of DCM/MeOH was increased from 70/30 to 80/20 w/w0% and the crystalline milvexian acetone solvated form P1.acetone was used instead of the crystalline Form A of milvexian.

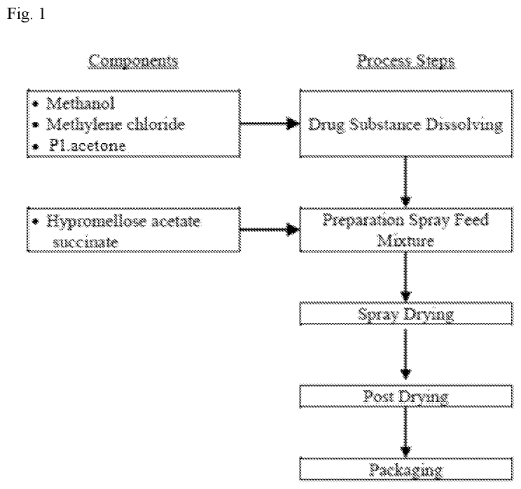

Based on the experiments above, the spray dried ASDP being a 3/1 milvexian/HPMC-AS MG formulation that is per g containing about 750 mg of milvexian free form (equivalent to 819.67 mg of crystalline P1.acetone solvated form of milvexian free form (acetone solvated form, equivalent to 750 mg of milvexian free form) per about 250 mg HPMC-AS MG was selected to be used for the manufacture of the tablets. The flow chart in FIG. 1 illustrates the complete spray drying process.

### Example 1g. Scale-Up of Spray Drying Process

About 12.3 wt. % of milvexian P1.acetone (equivalent to about 11.25 wt. % milvexian free form) and about 3.75 wt. % of HPMC-AS MG (AQOAT® AS-MG, Shin-Etsu Chemical Co., Ltd. (Niigata, Japan)) were mixed with a solvent mixture containing 80/20 w/w % DCM/MeOH. The milvexian P1.acetone and HPMC-AS MG dissolved quite immediately resulting in a light yellow clear solution of 400 kg. The clear solution at 21° C. was spray dried using a GEA PSD-3 spray dryer using the following parameter settings: Drying gas flow rate of 750 Kg/hr, atomization pressure of 28 Bar, feed rate at about 75 kg/h; inlet/outlet temperatures at about 98/45° C., condenser temperature of about −10° C. The spray drying process went on for about 1.5 hours and gave 12.4 kg of wet SDP. The wet SDP was subject to drying for 22 hours in a vacuum oven (Pink, Model VSD-650-650-140-7) at 40° C., with nitrogen flow, and a vacuum of approximately 200 mbar.

The produced powder had a median particle size distribution with a D_{v,50} of about 49 μm, span of about 1.7, and bulk/tapped density of 0.20/0.28 g/cm.

### Example 1h. Solubility of SDP Prepared in Example 1c

During this spray drying process, the crystalline acetone solvate (P1.acetone) form is converted into an amorphous free form as it's dissolved in the 80/20 w/w % DCM/MeOH solvent mixture followed by subsequent evaporation of all solvents to below ICH Q3C levels. Milvexian P1.acetone crystalline form is the preferred starting material for the manufacturing of the SDP instead of milvexian free form, due to its superior morphology and reliable crystallization process.

The aqueous solubility of the amorphous form (SDP) was found to be higher in comparison with the crystalline milvexian P1.acetone, as shown in Table 14. The dissolution rate of the amorphous form (amorphous solid dispersion-based spray-dried powder) of the drug substance was also significantly faster in human physiologically based aqueous media than the crystalline free form of the drug substance.

TABLE 14
Solubility of crystalline milvexian acetone
solvate and Milvexian SDP in Different Media

| | | Solubility as μg crystalline | | | |
|---|---|---|---|---|---|
| | | milvexian acetone solvate | | Solubility as μg amorph | |
| | | (P1.acetone) Dissolved per | | milvexian SDP Dissolved | |
| | | mL at 37° C. (μg/mL) | | per mL at 37° C. (μg/mL) | |
| Medium | Replicate | 1 day | 3 days | 1 day | 3 days |
| 0.1M HCl | 1 | 3.36 | 3.59 | 9.62 | 7.81 |
| | 2 | 3.47 | 3.30 | 9.60 | 8.65 |
| | 3 | 3.46 | 3.58 | 9.25 | 8.19 |
| | Average | 3.43 | 3.49 | 9.49 | 8.22 |
| | SD* | 0.06 | 0.16 | 0.21 | 0.42 |
| 0.05M sodium | 1 | 1.81 | 1.85 | 6.37 | 5.02 |
| acetate buffer | 2 | 1.69 | 1.95 | 5.66 | 4.38 |
| pH 4.5 | 3 | 1.57 | 1.85 | 5.41 | 4.15 |
| | Average | 1.69 | 1.88 | 5.81 | 4.51 |
| | SD* | 0.12 | 0.06 | 0.50 | 0.45 |
| 0.05M sodium | 1 | 1.53 | 1.56 | 31.14 | 4.76 |
| phosphate | 2 | 1.29 | 1.62 | 28.67 | 4.93 |
| buffer pH 6.8 | 3 | 1.48 | 1.62 | 34.07 | 5.89 |
| | Average | 1.43 | 1.60 | 31.29 | 5.19 |
| | SD* | 0.13 | 0.03 | 2.70 | 0.61 |

SD* = standard deviation

### Example 2A. Optimization of Weight Ratio of Binder to Filler

Initially, microcrystalline cellulose (MCC) and lactose were selected as excipients for their manufacturability, compressibility and flowability properties. Different MCC/lactose ratios were tested in this first screening study.

A dose of 25 mg of milvexian free form, with a 33 wt. % of solid amorphous dispersion particles was used for Ex.1, Ex.2, Ex.3 and Ex.4. See Table 15. Croscarmellose sodium was selected as a disintegrant in 5 wt. % by the total weight of the powder blend to allow fast disintegration of tablets (e.g. less than 2 minutes). Magnesium stearate was selected as lubricant in 1 wt. % by the total weight of the powder blend, considering continuous manufacturing (CM) requirements (e.g. capacity to feed low density material at high throughputs). The compositions of the Ex.1, Ex.2, Ex.3 and Ex.4 are provided in Table 19 below. The ingredients for the powder blends for Ex. Ex.2, Ex.3 and Ex.4 were blended using Turbula blender and compressed on the Courtoy Excentre single punch press (KCO1), equipped with punch set AC27/4 (6 mm round) at 300 kg (2.9 kN) compression force for Ex.1 and Ex.4, 550 kg (5.4 kN) for Ex.23 and 400 kg (3.9 kN) for Ex.3. Manufacturability (stickiness and tablet stability) was evaluated based on powder blend characteristics (visual observations) and tablet characteristics (weight (variability), hardness, disintegration time). In-process control (IPC) results for tablet properties and the powder blend manufacturability are summarized in Table 15 below.

TABLE 15
Direct Compressed Uncoated Tablet

| | Ex. 1 | Ex. 2 | Ex. 3 | Ex. 4 |
|---|---|---|---|---|
| Ingredient | Unit (mg) | Unit (mg) | Unit (mg) | Unit (mg) |
| SDP (Ex. 1f) | 33.333 | 33.333 | 33.333 | 33.333 |
| Microcrystalline cellulose * | 46.167 | 14.500 | 30.334 | 36.400 |
| Lactose monohydrate** | 14.500 | 46.167 | 30.333 | 24.267 |
| Croscarmellose sodium | 5.000 | 5.000 | 5.000 | 5.000 |
| Magnesium stearate | 1.000 | 1.000 | 1.000 | 1.000 |
| Microcrystalline cellulose/ | 75/25 | 25/75 | 50/50 | 60/40 |
| lactose monohydrate ratio | | | | |
| (w/w %) | | | | |
| Total Blend Weight | 100.000 | 100.000 | 100.000 | 100.000 |

Physical Properties

| | | | | |
|---|---|---|---|---|
| average disintegration time | 29.0 | 110 | 96 | 48 |
| (second, n = 6) | | | | |
| Thickness (mm) | 3.17 (1.64%) | 3.87 (0.79%) | 3.01 (0.60%)) | 3.10 (0.62%) |
| average (RSD %) | (n = 9) | (n = 3) | (n = 22) | (n = 17) |
| Average Hardness (N), | 76 | 79 ± 7 | 87 | 87 ± 11 |
| RSD % | (n = 1) | (n = 6) | (n = 1) | (n = 5) |
| Powder Blend Stickiness | blend adheres | No adherence | No adherence | No adherence |
| | to glass wall | to glass wall | to glass wall | to galss wall |
| Tableting Process | Robust | Not Robust, -, | Not Robust, - | Robust |
| Robustness | | manual | manual | |
| | | adjustment | adjustment | |
| | | needed | needed | |
| | | during | during | |
| | | filing | filing | |
| Average Tablet Weight | 101.0 ± 2.1 | 100.7 ± 1.1 | 102.2 ± 1.7 | 101.7 ± 1.5 |
| (mg) | (n = 10) | (n = 9) | (n = 22) | (n = 17) |

* Microcrystalline cellulose: MCC PH102 ®
**Lactose monohydrate
**Supertab 11SD ®

According to the results in Table 15 above, the powder blend of Ex.4 demonstrated excellent manufacturability, e.g., no observation of sticking on the wall, and the resulting tablet exhibited excellent stability (e.g., low variation of tablet weight and integrity). Thus, the weight ratio of 3:2 (60/40 w/w %) of binder (microcrystalline cellulose) to lactose monohydrate (filler) as for the composition of the tablet of Ex.4 was selected for further investigation. In addition, of the 5.0 wt. % of croscarmellose sodium and 1 wt. % of magnesium stearate are deemed suitable to achieve desired quality of tablets.

### Example 2B. Optimization of Binder

The binder in the composition of the Ex.4 with a weight ratio of MCC PH102 (binder) to lactose monohydrate (filler) at 60/40 (See Table 15 above) were further optimized. Microcrystalline cellulose PH102 in Ex.4 was replaced with silicified microcrystalline cellulose SMCC 90 and SMCC HD90 as exemplified in Ex.5, Ex.6 and Ex.7.

A dose of 25 mg of milvexian free form per 100 mg tablet weight (33 wt. % of spray dried SDP loading amount) was used for Ex.4, Ex. 5 and Ex.6. See Table 16. A dose of 25 mg of milvexian free form per 150 mg tablet weight (22 wt. % of spray dried SDP loading amount) was used for Ex.7. Croscarmellose sodium and magnesium stearate were used as a disintegrant and lubricant, respectively.

The compositions of the Ex.4, Ex.5, Ex.6 and Ex.7 are provided in Table 16 below. The ingredients in the compositions of Ex.4, Ex. 5 and Ex.6 (100 mg total tablet weight) were blended using Turbula blender and compressed on the Courtoy Exentre single punch press, equipped with punch set AC27/4 (6 mm round) at 300 kg (2.9 kN) compression force. The ingredients in the compositions of Ex.7 (150 mg total tablet weight) were blended and compressed on the Courtoy Excentre single punch press (KC01), equipped with punch set AC27/7 (7 mm round) at 350 kg (3.4 kN) compression force. Manufacturability was evaluated based on blend characteristics (visual inspection) and tablet characteristics (weight (variability), hardness, thickness, disintegration time). In-process control (IPC) results are shown in Table 16 below.

For Ex.4, Ex.5 and Ex.6 with a total tablet weight of 100 mg, only Ex.4 showed acceptable blend properties since sticking of blend on the wall was observed for Ex.5 and Ex.6. The powder blend of Ex.7, with a total weight of 150 mg, showed no sticking to the wall. Further, the resulting 150 mg tablet of Ex.7 exhibited quicker disintegration (13 seconds) as compared with that of the resulting 100 mg tablet of Ex.4 (48 seconds)

In light of the results for Ex.4, Ex.5, Ex.6 and Ex.7 as set forth in Table 16, the composition of Ex.7, which produces a 150 mg tablet (22 t. % SDP loading) (Ex.7, containing SMCC 90/lactose monohydrate in a 3:2 (60/40) ratio), was selected to achieve an increased tablet robustness and as the lead composition for further optimization. Further, SMCC 90 grade as binder and the weight ratio of SMCC 90 to lactose monohydrate at 3:2 (60/40) was selected for the solid pharmaceutical compositions of mivexian as described herein.

TABLE 16
Direct Compressed Uncoated Tablet

| | Ex. 4 | Ex. 5 | Ex. 6 | Ex. 7 |
|---|---|---|---|---|
| Ingredient | Unit (mg) | Unit (mg) | Unit (mg) | Unit (mg) |
| SDP (Ex.1f) | 33.333 | 33.333 | 33.333 | 33.333 |
| Microcrystalline cellulose* | 36.400 | — | — | — |
| Silicified microcrystalline | — | 36.4 | — | 64.6 |
| cellulose (SMCC 90) | | | | |
| Silicified microcrystalline | — | — | 36.4 | — |
| cellulose (SMCC HD90) | | | | |
| Lactose monohydrate** | 24.267 | 24.267 | 24.267 | 43.067 |
| Croscarmellose sodium | 5.000 | 5.000 | 5.000 | 7.500 |
| Magnesium stearate | 1.000 | 1.000 | 1.000 | 1.500 |
| binder/lactose | 60/40 | 60/40 | 60/40 | 60/40 |
| monohydrate ratio (w/w %) | | | | |
| Total Blend Weight (mg) | 100.000 | 100.000 | 100.000 | 150.000 |

Physical Properties

| | | | | |
|---|---|---|---|---|
| average disintegration time | 48 (n = 6) | 43 (n = 6) | 111 (n = 6) | 13 (n = 5) |
| (second) | | | | |
| Thickness (mm) | 3.10 (0.62%) | 3.11 (0.36%) | 3.01 (3.92%) | 3.37 (1.02%) |
| average (RSD %) | (n = 17) | (n = 16) | (n = 15) | (n = 13) |
| Average Hardness (N), | 87 ± 11.38 | 106 ± 13.71 | 111 ± 33.87 | 72 ± 5.82 |
| RSD % | (n = 5) | (n = 5) | (n = 8) | (n = 7) |
| Powder Blend Stickiness | No adherence | blend adheres | blend adheres | No adherence |
| | of blend to | to glass wall | to glass wall | of blend to |
| | glass wall | | | glass wall |
| Tableting Process | Robust | Robust | Robust | Robust |
| Robustness | | | | |
| Average Tablet Weight | 101.7 ± 1.5 | 100.9 ± 1.8 | 102.8 ± 1.8 | 148.05 ± 1.7 |
| (mg) | (n = 17) | (n = 22) | (n = 23) | (n = 13) |

*Microcrystalline cellulose: MCC PH102 ®
**Lactose monohydrate
**Supertab 11SD ®

### Example 2C. Optimization of Lactose Grade

From previous experience, it is known that the use of lactose monohydrate (lactose Supertab) might result in issues during physiology based dissolution testing (PBDT). Occurrences have been observed were lactose Supertab entraps solid drug product in the PBDT dissolution bath. To evaluate this possible effect of lactose grade, a new formulation Ex.8 containing lactose Tablettose was prepared. The compositions of the Ex.7 and Ex.8 are provided in Table 17 below of which different grades of lactose were used.

TABLE 17
Direct Compressed Uncoated Tablet

| | Ex. 7 | Ex. 8 |
|---|---|---|
| Ingredient | Unit (mg) | Unit (mg) |
| SDP (Ex. 1f) | 33.333 | 33.333 |
| Silicified microcrystalline | 64.6 | 64.6 |
| cellulose (SMCC 90) | | |
| Lactose monohydrate | 43.067 | — |
| Supertab 11SD ® | | |
| Lactose monohydrate | — | 43.067 |
| Tablettose ®* | | |
| Croscarmellose sodium | 7.500 | 7.500 |
| Magnesium stearate | 1.500 | 1.500 |
| binder/lactose monohydrate | 60/40 | 60/40 |
| ratio (w/w %) | | |
| Total Blend weight (mg) | 150.000 | 150.000 |

Powder blend Physical Properties

| | | |
|---|---|---|
| LOD (%) | 2.89 | 2.94 |
| Angle of Response (°) | 50.17 | 51.09 |
| Bulk Density (g/mL) | 0.47 | 0.39 |
| Tapped Density (g/mL) | 0.56 | 0.48 |
| Hausner Index | 1.21 | 1.23 |
| Carr's Index | 17.14 | 18.54 |
| FFC (ring shear) | 10.28 | 10.84 |

*Lactose monohydrate Tablettose ®
*Tablettose ® 80 or Tablettose ® 100 is manufactured by MEGGLE GmbH & Co. KG, Germany. Tablettose ® 80 was used in Ex. 8.

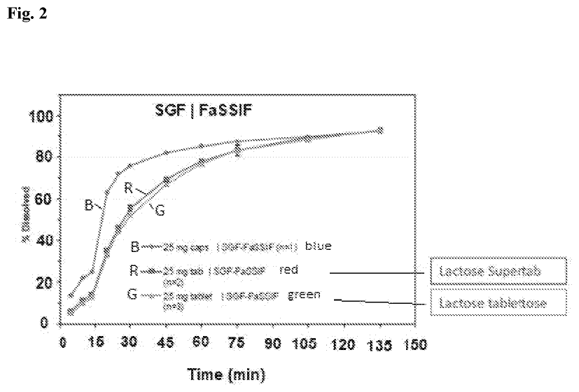

Both Ex.7 and Ex.8 were evaluated for PBDT and blending properties. Both Ex.7 and Ex.8 had a similar PBDT profile, insinuating that the lactose grade does not have a significant influence on the PBDT dissolution profile. See FIG. 2.

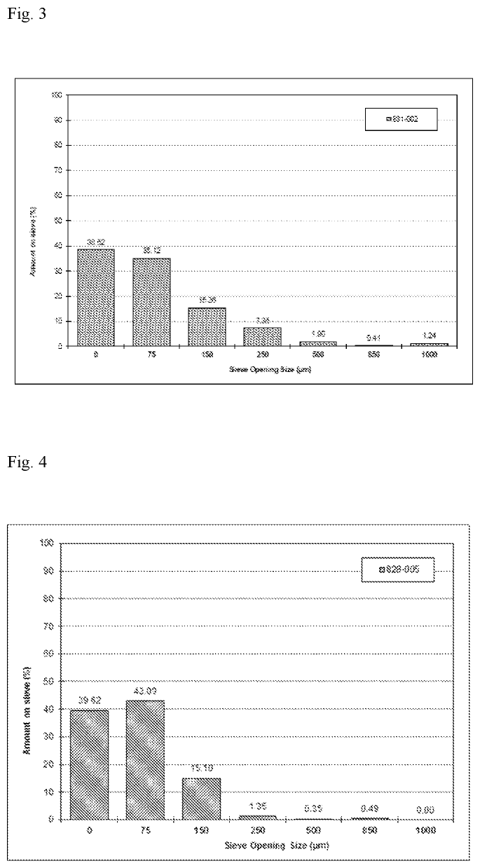

However, the blend properties of Ex.8 were less favorable compared to those of Ex.7 (see Table 17 and FIGS. 3-4). The bulk and tapped density of Ex.8 was lower than that of Ex.7, which could result in potential less favorable manufacturability when continuous manufacturing mode is used for tableting due to a too low density to be able to feed by the feeders at high throughputs. Taking into account the similar PBDT profiles and the more favorable blend properties of Ex.7, the lactose grade Supertab 11 SD is selected the solid pharmaceutical compositions of milvexian as described herein.

### Example 2D. Optimization of Disintegrant

To investigate different types of disintegrants, the lead formulation (Ex.7) using croscarmellose sodium, was compared to Ex.9 and Ex.10 containing L-hydroxypropyl cellulose (L-HPC) and crospovidone, respectively, as disintegrant. The compositions of the Ex.7, Ex.9, and Ex.10, as well as the powder blending and compression equipment, and parameters are provided in Table 18 below.

Three different powder blends according to the compositions provided in Table 18 below were prepared, each with a different disintegrant (Ex.7 with croscarmellose sodium. Ex.9 with L-HPC, and Ex. 10 with crospovidone). Based on previous screening studies on formulations as set forth in Tables 15-17 above, SMCC 90 was selected as binder and lactose monohydrate Supertab 11SD was used as filler (SMCC 90/lactose monohydrate Supertab® 11SD in a weight ratio of 3:2 (60/40), and a dose of 25 mg of milvexian free form per 150 mg total tablet weight was applied (resulting in 22% SDP loading).

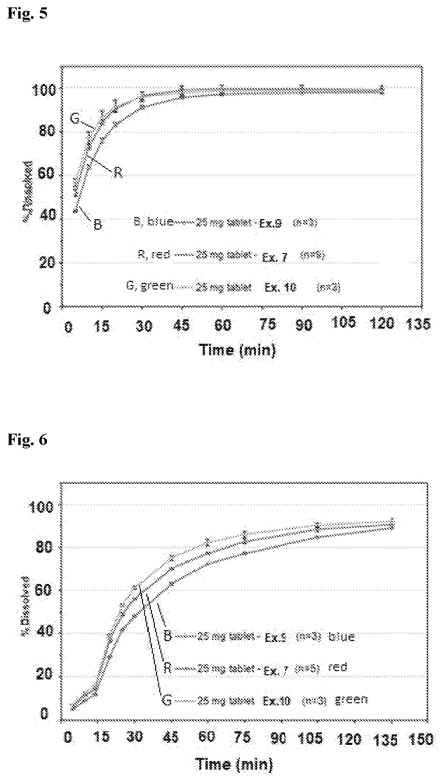

Manufacturability was evaluated based on blend characteristics (visual inspection) and tablet characteristics (weight (variability), hardness, thickness, disintegration time) (Table 18 below). The dissolution profiles of Ex.7, Ex.9, and Ex.10 were compared per dosage strength (FIG. 5 and FIG. 6).

TABLE 18
Direct Compressed Uncoated Tablet

| | Ex. 7 | Ex. 9 | Ex. 10 |
|---|---|---|---|
| Ingredient | Unit (mg) | Unit (mg) | Unit (mg) |
| SDP (Ex.1f) | 33.333 | 33.333 | 33.333 |
| Silicified microcrystalline | 64.6 | 64.6 | 64.6 |
| cellulose (SMCC 90) | | | |
| Lactose monohydrate* | 43.067 | 43.067 | 43.067 |
| Croscarmellose sodium | 7.500 | — | — |
| hydroxypropyl cellulose | — | 7.5 | — |
| (L-HPC) | | | |
| crospovidone | — | — | 7.5 |
| Magnesium stearate | 1.500 | 1.000 | 1.000 |
| binder/lactose | 60/40 | 60/40 | 60/40 |
| monohydrate ratio (w/w %) | | | |
| Total Blend Weight (mg) | 150.000 | 150.000 | 150.000 |

Blending and Compression Equipment and Operation Parameters

| | | | |
|---|---|---|---|
| Blender | Turbula blender | Turbula blender | Turbula blender |
| Press | Courtoy Excentre | Killian Styl'One | Killian Styl'One |
| | single punch press | single punch | single punch tablet |
| | (KC01) | tablet press | press (KC22) |
| | | (KC22) | |
| Punch | punch set AC27/7 | punch set P137 | punch set P137 |
| | (7 mm round) | (7 mm round) | (7 mm round) |
| Compression Force | 350 kg (3.4 kN) | 4 kN | 4 kN |

Physical Properties

| | | | |
|---|---|---|---|
| average disintegration time | 13 (n = 5) | 20 (n = 2) | 10 (n = 3) |
| (second) | | | |
| Thickness (mm) | 3.37 (1.02%) | 4.08 (0.26%) | 4.09 (0.90%) |
| average (RSD %) | (n = 13) | (n = 8) | (n = 8) |
| Average Hardness (N), | 72 ± 5.82 | 94 ± 2.80 | 98 ± 4.18 |
| RSD % | (n = 7) | (n = 8) | (n = 8) |
| Tableting Process | Robust/No | Robust, similar | Robust |
| Robustness | adherence of blend | to Ex. 7 | |
| | to glass wall | | |
| Average Tablet Weight | 148.05 ± 1.7 | 155 ± 0.7 | 152 ± 1.8 |
| (mg) | (n = 13) | (n = 8) | (n = 8) |

Lactose monohydrate
** Supertab 11SD ®

For Ex.9 (containing L-HPC), a slightly higher disintegration time result and slower dissolution profile was observed compared to Ex. 7 (containing croscarmellose sodium). For Ex.10 (containing crospovidone), similar dissolution profile, blend, and tablet properties were observed compared to Ex.7 (containing croscarmellose sodium). Based on these results as in Table 18 above, croscarmellose sodium was used as disintegrant.

It can be concluded from previous screening studies that Ex.7 can be selected as lead composition for milvexian free form 25 mg tablet. The composition for scale up manufacture is shown in Table 19.

TABLE 19
Quantitative Composition of Lead Composition
for Milvexian free form 25 mg Tablet

| Ingredient | unit (mg) | % w/w |
|---|---|---|
| SDP (Ex. 1f) | 33.333 | 22.222 |
| SMCC 90 | 64.600 | 43.067 |
| Lactose Monohydrate (Supertab 11SD) | 43.067 | 28.711 |
| Croscarmellose sodium | 7.500 | 5.000 |
| Magnesium stearate | 1.500 | 1.000 |
| Total | 150.000 | |

### Example 3. Scale Up Direct Compression Manufacture Process for 25 mg and 100 mg Film-Coated Tablets of Milvexian

### Example 3A

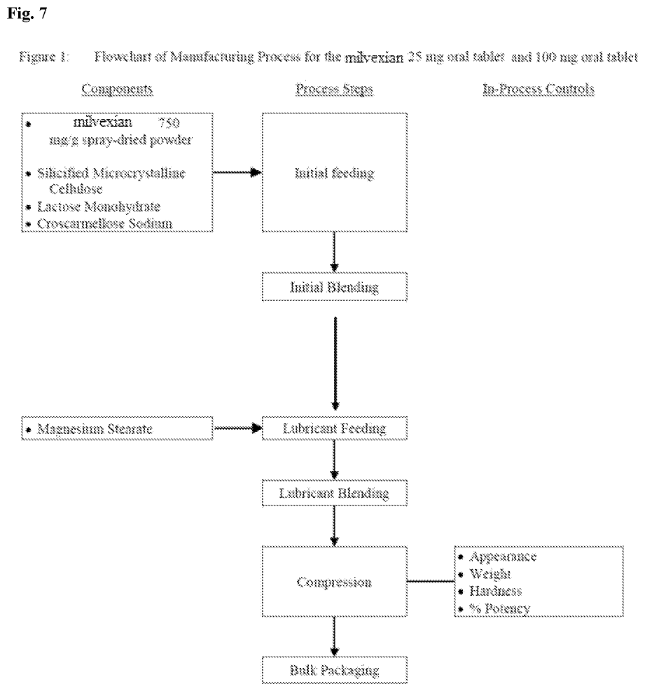

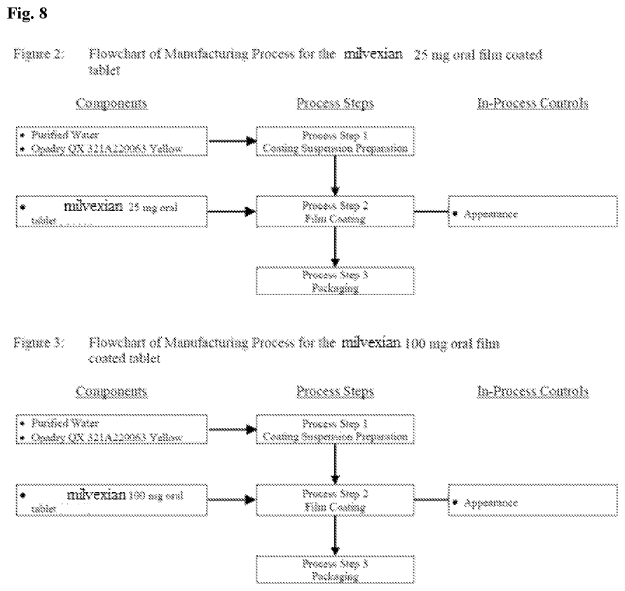

The manufacturing process flow chart of milvexian 25 mg oral tablet core and 100 mg oral tablet core is given in FIG. 7. The manufacturing process flow chart of milvexian 25 mg oral film-coated tablet and 100 mg oral film-coated tablet is given in FIG. 8. Thus, FIGS. 7 and 8 together set forth the manufacturing process for the milvexian tablets.

In this study, the dose proportionality of the lead composition (refer to Table 19) was evaluated. One blend (with a 22% SDP load) was prepared according to the composition presented in Table 19. This blend was then used to make 4 dose proportional tablet compositions with a dose of 25, 50, 75, and 100 mg of milvexian free form per 150, 300, 450, and 600 mg total tablet weight, respectively. An overview of the compositions tested is shown in Table 20.

TABLE 20
Overview Compositions Used in Composition Screening

| Ex. | strength | Tablet core | Punch set |
|---|---|---|---|
| number | (mg) | weight (mg) | (mm) |
| Ex. 7 | 25 | 150 | 7 |
| Ex. 11 | 50 | 300 | 9 |
| Ex. 12 | 75 | 450 | 10 |
| Ex. 13 | 100 | 600 | 11 |

From the same blend (Table 19 above), the four compositions were prepared using the manufacturing equipment and compression forces presented in Table 21.

TABLE 21
Blending and Compression Equipment and Parameters

| Ex. | | | Compression |
|---|---|---|---|
| number | Press | Punch | Force |
| Ex. 7 | Courtoy Excentre single | punch set AC27/7 | 470 kg |
| (25 mg) | punch press (KC01) | (7 mm round) | (4.6 kN) |
| Ex. 11 | Courtoy Excentre single | punch set AC27/13 | 400 kg |
| (50 mg) | punch press (KC01) | (9 mm round) | (3.9 kN) |
| Ex. 12 | Courtoy Excentre single | punch set AC27/17 | 500 kg |
| (75 mg) | punch press (KC01) | (10 mm round) | (4.9 kN) |
| Ex. 13 | Courtoy Excentre single | Punch set AC27/19 | 550 kg |
| (100 mg) | punch press (KC01) | (11 mm round) | (5.4 kN) |

Manufacturability was evaluated based on blend characteristics (sticking/static tendencies, flow, segregation tendencies) and tablet characteristics (weight (variability), hardness, thickness, disintegration time). Blend IPC results and tablet IPC results are shown in Table 22 and Table 23. FIG. 4 shows the blend particle size distribution.

TABLE 22
SDP Powder Blend in-process control (IPC) Results

| Parameter | IPC results |
|---|---|
| LOD | 2.89% |
| Angle of repose | 50.17° |
| Bulk density | 0.47 g/mL |
| Tapped density | 0.56 g/mL |
| Hausner index | 1.21 |
| Carr's index | 17.14 |
| FFC (ring shear) | 10.28 |

TABLE 23
Tablet Core in-process control (IPC) Results

| Parameter | Ex. 7 | | Ex. 14 | | Ex. 15 | | Ex. 16 | |
|---|---|---|---|---|---|---|---|---|
| Disintegration | 18 | | 9 | | 13 | | 14 | |
| time (seconds, | | | | | | | | |
| n = 1) | | | | | | | | |
| average (RSD %) | | | | | | | | |
| Hardness (N, | 111 | (2.77%) | 92 | (6.60%) | 105 | (3.08%) | 112 | (4.71%) |
| n = 10) | | | | | | | | |
| average (RSD %) | | | | | | | | |
| Thickness (mm, | 3.21 | (0.21%) | 4.77 | (0.31%) | 5.71 | (0.52%) | 6.37 | (0.11%) |
| n = 10) | | | | | | | | |
| average (RSD %) | | | | | | | | |
| Weight (mg, | 152.7 | (0.81%) | 301.4 | (0.29%) | 452.6 | (0.22%) | 597.1 | (0.18%) |

| | | | | |
|---|---|---|---|---|
| n = 10) average | | | | |
| (RSD %) | | | | |
| Tableting | Ok | Ok | Ok | Ok |
| Process | | | | |
| Robustness | | | | |

Based on blend and tablet IPC results, it can be concluded that the composition of the lead composition for milvexian free form 25 mg tablet (refer to Table 19 above) is suitable for manufacturing dose proportional tablets.

In this experiment, a round punch was used for all tablet strengths. For the 100 mg tablet, this resulted in an 11 mm round tablet which was not preferred for patient compliance (swallowability issues in elderly population). Therefore, an oblong tablet shape was evaluated for the 100 mg dose tablet.

These tablets were compressed on the Excentre single punch tablet press, equipped with punch set AC27/67 (16.6 mm×8 mm oblong) at 650 kg (6.4 kN) compression force. Manufacturability was evaluated based on blend characteristics (visual evaluation) and tablet characteristics (weight (variability), hardness, thickness, disintegration time). IPC results are shown in Table 24.

TABLE 24
Tablet IPC Results of Milvexian free form 100 mg Tablet

| Parameter | 100 mg Tablet | |
|---|---|---|
| Disintegration time (sec, | NT | |
| n = x) | | |
| Hardness (N, n = x) | 158 | (4.35%) |
| Thickness (mm, n = x) | 6.21 | (0.33%) |
| Weight (mg, n = x) | 595.9 | (0.31%) |

| | |
|---|---|
| Tableting Process | Ok |
| Robustness | |

NT = Not tested

Based on these results, the 100 mg milvexian free form tablet formula is considered suitable for change of shape from round to oblong.

Screening for a disintegrant, as was done for milvexian free form 25 mg tablet, was repeated for milvexian free form 100 mg tablet formula. This resulted in selection of croscarmellose sodium as disintegrant as well, leading to the selection of the milvexian free form 100 mg tablet with the same blend as the lead 25 mg tablet as lead compositions. Both lead compositions is presented together in Table 25 below.

TABLE 25
Composition of Milvexian free form
25 mg and 100 mg Lead Compositions

| | Ex. 11 | Ex. 12 | |
|---|---|---|---|
| | Unit Dose Strength | | |
| | 25 mg | 100 mg | |
| | unit (mg) | unit (mg) | wt. % |
| SDP(Ex. 1f) | 33.333 | 133.333 | 22.222 |
| SMCC 90 | 64.600 | 258.400 | 43.067 |
| Lactose Monohydrate | 43.067 | 172.267 | 28.711 |
| (Supertab 11SD) | | | |
| Croscarmellose | 7.500 | 30.000 | 5.000 |
| sodium | | | |
| Magnesium stearate | 1.500 | 6.000 | 1.000 |
| Total Weight | 150.000 | 600.000 | |

### Example 3B. Scale Up Manufacturing Process for the Film Coated Tablet by Direct Compression

The manufacturing process flow chart of the milvexian 25 mg oral tablet core (Ex.11) and 100 mg oral tablet core (Ex. 12) is given in Table 25 and FIG. 7.

The manufacturing process flow chart of the milvexian 25 mg oral film-coated tablet (Ex.17) and 100 mg oral film-coated tablet (Ex. 18) is given in Table 26 and FIG. 8.

Various continuous manufacture runs using the optimized process described in Example 3B have showed a robust continuous manufacture process towards flow, CU, hardness and weight (see back up slides). Robust Content uniformity obtained throughout development campaign: RSD 0.8%-2.2% with 11 CM runs for both 25 mg and 100 mg. Robust weight obtained for both strengths throughout development campaign: RSD 0.5%-2% with 11 CM runs for both 25 mg and 100 mg. Robust tablet hardness within IPC limits irrespective of spray drying scale, spray drying conditions and CM conditions (mixing & lubrication speed and line throughput).

TABLE 26
Film-coated Tablet of Milvexian at 25 mg and 100 mg Strength

Amount

| | | Amount | Amount |
|---|---|---|---|
| | | per dose | per dose |
| | | (25 mg, | (100 mg, |
| Component | wt. % | Ex. 17) | Ex. 18) |
| SDP (Ex. 1f) | 22.22 | 33.33 | 133.3 |
| Silicified Microcrystalline | 43.07 | 64.6 | 258.4 |
| cellulose | | | |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 | 3.0 | 4.5 | 18.0 |
| Yellow | | | |
| | | 154.5 | 618 |

### Example 3C. Film-Coated Tablet of 100 mg Per Dose of Milvexian Prepared by Roller Compaction

The SDPs prepared according to Example 1e. above was used to prepare film-coated tablet of 100 mg per dose of milvexian by dry granulation and roller compaction method (RC tablet. Ex. 19). The composition of the RC tablet Ex, 19 is given in Table 27 below.

TABLE 27
Film-Coated Tablet of 100 mg per dose of
Milvexian Prepared by Roller Compaction

| | Ex. 19 (100 mg dose) |
|---|---|
| Component | Unit (mg) |
| Intragranular | |
| SDP (Ex. 1f) | 133.3 |
| Microcrystalline cellulose | 206.267 |
| Croscarmellose sodium | 20.0 |
| Magnesium stearate | 4.0 |
| Total Intragranular | 485 |
| Extragranular | |
| Silicified microcrystalline | 142.267 |
| cellulose (SMCC) | |
| Lactose monohydrate | 71.133 |
| Croscarmellose sodium | 20.0 |
| Magnesium stearate | 3.0 |
| Weight ratio of MCC + SMMC to | 4.9:1 |
| lactose monohydrate | |
| Total | 600 |

### Example 4. Dissolution Tests and Stability Study on Ex. 17 and Ex. 18

Stability Tests and Results. The shelf stability for all climatic zones were performed on the drug product Ex. 17 and Ex. 18 in high density polyethylene (HDPE) bottle with 2 gram silica at 25° C./60% RH and 30° C./75% RH. No change observed for appearance. No degradation impurity observed by HPLC assays. No indication of crystalline API formation in the drug product by XPRD. The film-coated tablet for 25 mg and 100 mg strength milvexian is stable at 25° C./60% RH and 30° C./75% RH up to at least 24 months.

In routine dissolution tests using paddle apparatus (USP type 2), plateau shows no indication of crystalline API formed in the drug product. In addition, solid state NMR showed no indication of crystalline API formation in drug product store in the HDPE bottle for 22 months.

### Dissolution Tests and Results

The dissolution test was performed in 900 mL of dissolution medium at 37.0° C. using Paddle Apparatus (USP type 2, Ph.Eur., JP.) at a rotation speed of 75 rpm. Samples are removed after 5, 10, 15, 20, 30, 45, 60, 90 and 120 minutes from test initiation and analyzed for milvexian by UHPLC at 220 nm UV. 0.05 M acetate buffer pH 4.5 with 0.2% (w/v) sodium lauryl sulfate (SLS) solution has been used as dissolution medium during formulation development. A role of SLS (surfactant) in the dissolution medium is a wetting aid to facilitate complete dissolution of milvexian from tablets, rather than to increase the solubility of milvexian. Dissolution data from both tests are included in this disclosure and unless otherwise specified, the results reported are average of values from six tablets

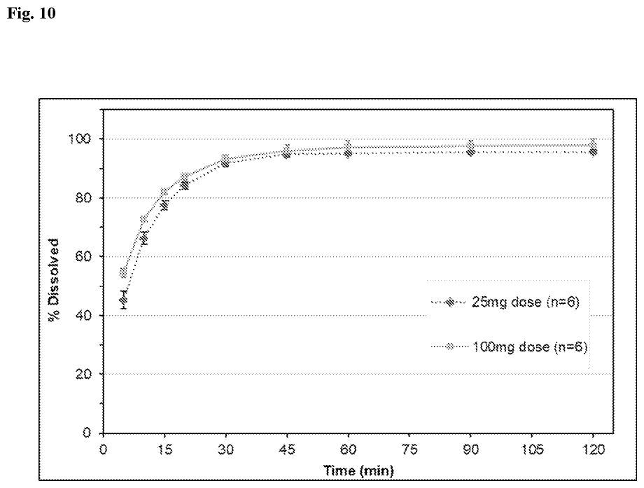

Dissolution tests using standard USP method as described above was performed on the 25 mg and 100 mg tablet of Ex.17 and Ex. 18 are summarized in Table 28 below. The results are also illustrated in FIG. 10.

TABLE 28
Dissolution Profiles for Ex. 17 and Ex. 18

| Time | Ex. 17 (25 mg) | Ex. 18 (100 mg) |
|---|---|---|
| (min) | % dissolved | % dissolved |
| 5 | 45 | 54 |
| 10 | 66 | 73 |
| 15 | 77 | 82 |
| 20 | 84 | 87 |
| 30 | 92 | 93 |
| 45 | 95 | 96 |
| 60 | 95 | 97 |
| 90 | 96 | 98 |
| 120 | 96 | 98 |

### Example 5. Bioavailability Study of Ex. 17 and Ex. 18 Against Compara. Ex. 1 and Compara. Ex. 2 in Healthy Participants and Results

The first Phase 1 trial is an open-label, randomized, crossover study to evaluate the relative oral bioavailability, pharmacokinetics, and food effect after single dose (for Part 1, Part 3, and Part 4) or multiple-dose (for Part 2). Part 1 of this first Phase 1 study is to evaluate the relative bioavailability and food effect of a single dose of 200 mg milvexian administered as film coated DC tablet Ex. 18 and film-coated RC tablet Ex. 19 compared with comparative SDP oral capsule Compara. Ex. 1 under fasting and fed conditions. Part 2 of this first Phase 1 study is to characterize the pharmacokinetic (PK) of multiple twice daily administered doses for 5 days of milvexian administered as Ex. 18 and Compara. Ex. 1 or Compara. Ex. 2 (SDP oral capsules) at 25 mg or 200 mg. The Compara. Ex. 1 and Compara. Ex. 2 capsule formulations (see Table 29 below) are described in WO 2020210629 of which the capsule comprises MCC and lactose anhydrous DC in a weight ratio of 1:1 binder (MCC) to filler (lactose anhydrous).

Blood samples were drawn at predetermined time points following drug administration as specified in the clinical study protocols. Concentration of the samples are measured using a validated analytical method (Liquid Chromatography with Tandem Mass Spectroscopy). Individual subject pharmacokinetic parameters (e.g. Cmax, AUCₗₐₛₜ, and AUC_{∞}) are derived by non-compartmental methods using Phoenix™ WinNonlin® (version 8. 1, Pharsight, A Certara™ Company, L.P., Princeton, NJ. USA) software from the time-concentration profiles.

TABLE 29
SDP Capsule Formulation Composition
of Compara. Ex. 1 and Compara. Ex. 2

| | Compara. Ex. 1 | Compara. Ex. 2 |
|---|---|---|
| Component | (100 mg dose) | (25 mg dose) |
| Intragranular | | |
| SDP of 75% Compound | 133.3 | 33.33 |
| (I):HPMCAS* | | |
| Microcrystalline cellulose | 167.7 | 41.925 |
| (MCC) | | |
| Lactose anhydrous DC | 167.7 | 41.925 |
| Weight ratio of MCC to lactose | 1:1 | 1:1 |
| anhydrous | | |
| Croscarmellose sodium | 12.5 | 3.125 |
| Colloidal silicon dioxide | 2.50 | 0.625 |
| Magnesium stearate | 1.25 | 0.3125 |
| Total Intragranular | 485 | 121 |
| Extragranular | | |
| Croscarmellose sodium | 12.5 | 3.125 |
| Magnesium stearate | 2.5 | 0.625 |
| Total | 500 | 125 |

*SDP prepared according to the composition and method described in WO 2020210629

The treatment regimens for Part 1 and Part 2 are summarized in Table 30 below.

TABLE 30
Treatment Regimen used in Part 1 and Part 2

| Treatment | Formulation | Dose | Food |
|---|---|---|---|
| A | 100 mg film-coated DC* | 200 mg (2 × 100 mg) | Fasted |
| (Part 1A) | oral tablet (Ex. 18) | | |
| B | 100 mg film-coated RC** | 200 mg (2 × 100 mg) | Fasted |
| (Part 1A) | oral tablet (Ex, 19) | | |
| C | 100 mg SDP oral capsule | 200 mg (2 × 100 mg) | Fasted |
| (Part 1A) | (Compara. Ex. 1) | | |
| D | 100 mg film-coated DC* | 200 mg (2 × 100 mg) | Fed |
| (Part 1B) | oral tablet (Ex. 18) | | |
| E | 100 mg film-coated RC** | 200 mg (2 × 00 mg) | Fed |
| (Part 1B) | oral tablet (Ex, 19) | | |
| F | 100 mg SDP oral capsule | 200 mg (2 × 100 mg) | Fed |
| (Part 1B) | (Compara. Ex. 1) | | |
| G | 100 mg film-coated DC* | 200 mg (2 × 100 mg) | BID for |
| (Part 2A) | oral tablet (Ex. 18) | | 5 days |
| H | 100 mg SDP oral capsule | 200 mg (2 × 100 mg) | BID for |
| (Part 2A) | (Compara. Ex. 1) | | 5 days |
| I | 25 mg film-coated DC* | 25 mg (1 × 25 mg) | BID for |
| (Part 2B) | oral tablet (Ex. 17) | | 5 days |
| J | 25 mg SDP oral capsule | 25 mg (1 × 25 mg) | BID for |
| (Part 2B) | Compara. Ex. 2 | | 5 days |

*DC: direct compression
**RC: roller compaction

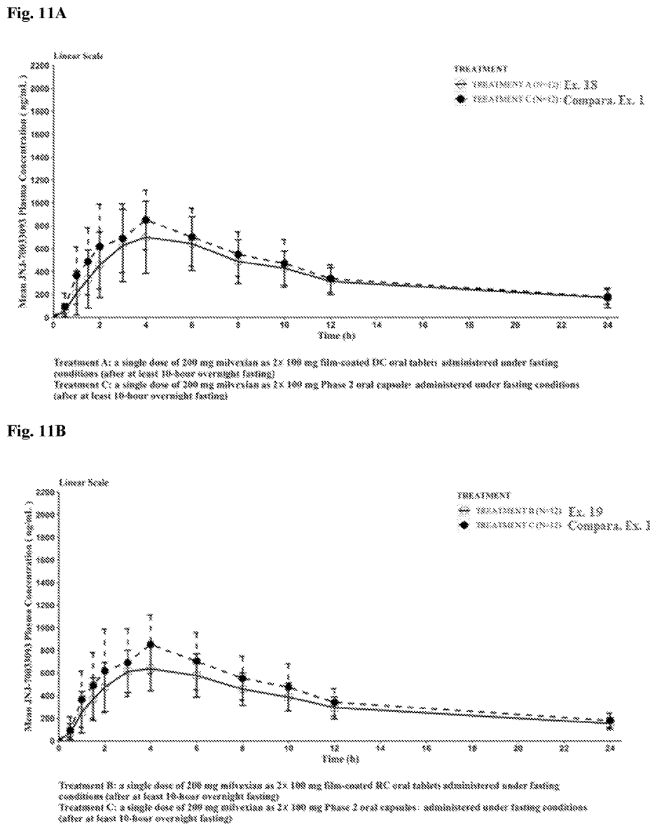

The results of Part 1 study are summarized in Tables 31-32 and FIGS. 11A-B and 12A-B.

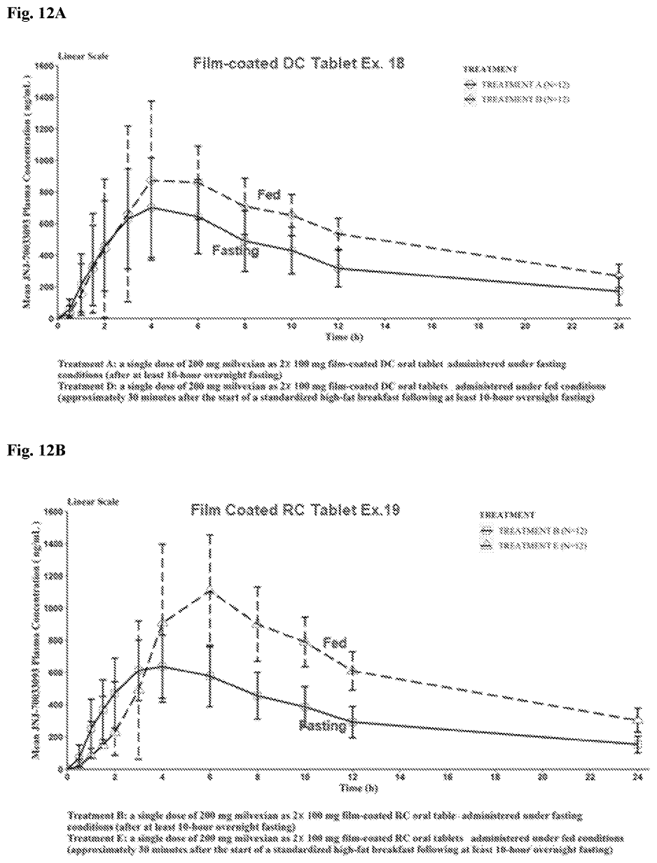

In Part 1 of single dose administration regimen, Ex. 18 (DC tablet) shows about 9.0% to about 11% and Ex. 19 (RC tablet) shows about 15% about 20% lower bioavailability, as compared to the Compara. Ex. 1 (SDP capsule) at 200 mg dose. Ex. 18 (DC tablet) shows about 20-40% higher exposure with food, in comparison Ex. 19 (RC tablet) shows about 60-80% higher exposure with food, at 200 mg dose. Ex. 18 (DC tablet) shows about 42% higher exposure [AUCinf] with food, in comparison Ex. 19 (RC tablet) shows about 76% higher exposure [AUCinf] with food, at 200 mg dose. Ex. 18 (DC tablet) has lower food effects. To achieve better patient compliance, it is preferred to have milvexian being administered with or without food. A drug formulation with small food effects provides better patient compliance. The results in FIGS. 12A and B demonstrated that DC tablet Ex. 18 performed better than the RC tablet Ex. 19 because of smaller food effects.

Table 31 summarized statistical results for estimated ratio of means and 90% confidence interval (90% CI) for milvexian: Treatment A/Treatment C: Pharmacokinetics Data Analysis Set (FIGS. 11A-11B).

TABLE 31
rBA-DC tablets vs capsule (2 × 100 mg, fasted)

| | | Numbers | | | | |
|---|---|---|---|---|---|---|
| | | of | Geometric Means | | Ratio of | |
| | | participants | Treatment C | Treatment | Geometric | 90% |
| Substance | PK data | (N) | (Comparator) | A (Test) | Means (%) | CI (%) |
| Milvexian | Cₘₐₓ | 12 | 860 | 770 | 89.53 | 77.91-102.88 |
| | (ng/mL) | | | | | |
| | AUCₗₐₛₜ | 12 | 12077 | 10834 | 89.71 | 79.52-101.21 |
| | (h*ng/mL) | | | | | |
| | AUC_{0-inf} | 11 | 11894 | 10882 | 91.49 | 80.50-103.99 |
| | (h*ng/mL) | | | | | |

Note:
Analysis done on log transformed data and the results were back-transformed using anti-logarithm.
Mixed-effect ANOVA model included log transformed PK parameters as response variable, fixed effect term as treatment, treatment period and treatment sequence and participant as random effect.
Participants who completed all 3 treatment periods with estimable pharmacokinetics parameters are only included in the analysis.
Treatment A: a single dose of 200 mg milvexian as 2 × 100 mg film-coated DC oral tablets (Ex. 17) administered under fasting conditions
Treatment C: a single dose of 200 mg milvexian as 2 × 100 mg Compara. Ex. 1 (SDP oral capsules) administered under fasting conditions

Table 32 summarized statistical results for estimated ratio of means and 90% confidence interval (90% CI) for milvexian, sensitivity analysis: Treatment D/Treatment A; Pharmacokinetics Data Analysis Set (FIGS. 12A and 12B below)

TABLE 32
Food Effects on DC film-coated tablet (2 × 100 mg)

| | | | | Geometric | | | |
|---|---|---|---|---|---|---|---|
| | | Numbers of | | Means | Geometric | Ratio of | |
| | | participants (N) | | Treatment | Means | Geometric | |
| | | Treatment A | Treatment | A | Treatment | Means | 90% |
| Substance | PK data | (Comparator) | D (Test) | (Comparator) | D (Test) | (%) | CI (%) |
| Milvexian | Cₘₐₓ | 12 | 12 | 770 | 988 | 128.38 | 104.77-157.32 |
| | (ng/mL) | | | | | | |
| | AUCₗₐₛₜ | 12 | 12 | 10834 | 15571 | 143.71 | 120.55-171.33 |
| | (h*ng/mL) | | | | | | |
| | AUC_{0-inf} | 12 | 12 | 11117 | 15815 | 142.27 | 119.28-169.69 |
| | (h*ng/mL) | | | | | | |

Note:
Analysis done on log transformed data and the results were back-transformed using anti-logarithm.
The ANOVA model included log transformed PK parameters as response variable and fixed effect term as treatment.
Participants who have taken at least one dose of study drug are included in the analysis.
Treatment A: a single dose of 200 mg milvexian as 2 × 100 mg film-coated DC oral tablets (Ex. 18) administered under fasting conditions
Treatment D: a single dose of 200 mg milvexian as 2 × 100 mg film-coated DC oral tablets (Ex. 18) administered under fed conditions

In Part 2 of multiple dose BID administration regimen, at 200 mg, Ex. 18 (DC tablet) shows about 5-7%~lower bioavailability, as compared to the Compara. Ex. 1 (SDP capsule). At 25 mg, Ex. 17 (DC tablet) shows about 11-1l3% lower bioavailability, as compared to the Compara. Ex. 2 (SDP capsule).

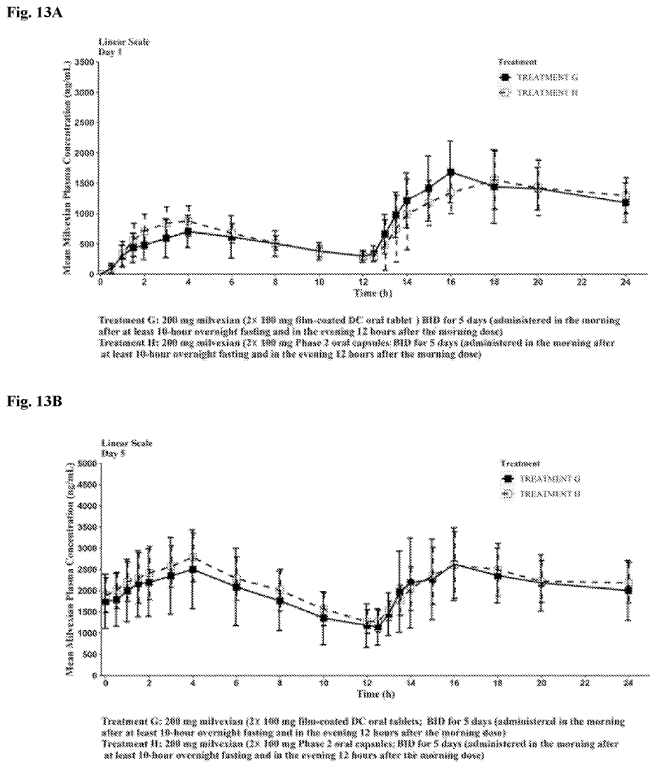

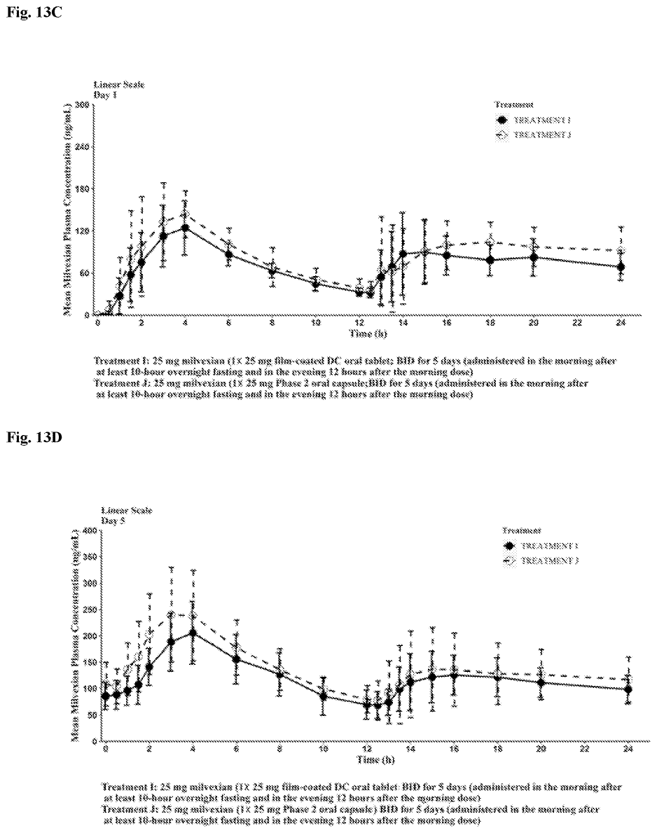

The results are summarized in Table 33-34 and FIGS. 13A-13D. Table 33 summarized statistical results for estimated ratio of means and 9000 confidence interval for milvexian: Treatment G/Treatment H, Pharmacokinetics Data Analysis Set.

TABLE 33

| | | Numbers | | | | |
|---|---|---|---|---|---|---|
| | | of | Geometric Means | | Ratio of | |
| | PK | participants | Treatment H | Treatment | Geometric | 90% CI |
| Substance | Parameter | (N) | (Comparator) | G (Test) | Means (%) | (%) |
| Milvexian | Cₘₐₓ | 12 | 2765 | 2614 | 94.55 | 87.77-101.84 |
| | (ng/mL) | | | | | |
| | AUCₗₐₛₜ | 12 | 56164 | 52207 | 92.95 | 85.76-100.75 |
| | (h*ng/mL) | | | | | |
| | AUC_{∞} | 12 | 56753 | 52751 | 92.95 | 85.69-100.82 |
| | (h*ng/mL) | | | | | |

Treatment G: 200 mg milvexian (2 × 100 mg film-coated DC oral tablets; Ex. 18) BID for 5 days (administered in the morning after at least 10-hour overnight fasting and in the evening 12 hours after the morning dose)
Treatment H: 200 mg milvexian (2 × 100 mg SDP capsule; Compara. Ex. 1) BID for 5 days (administered in the morning after at least 10-hour overnight fasting and in the evening 12 hours after the morning dose)

Table 34 summarized statistical results for estimated ratio of means and 90% confidence interval (90% CI) for milvexian: Treatment I/Treatment J; Pharmacokinetics Data Analysis Set

TABLE 34

| | | Numbers | | | | |
|---|---|---|---|---|---|---|
| | | of | Geometric Means | | Ratio of | |
| | PK | participants | Treatment J | Treatment I | Geometric | 90% |
| Substance | Parameter | (N) | (Comparator) | (Test) | Means (%) | CI (%) |
| Milvexian | Cₘₐₓ | 12 | 157 | 137 | 87.77 | 77.36-99.59 |
| | (ng/mL) | | | | | |
| | AUCₗₐₛₜ | 12 | 2874 | 2556 | 88.91 | 84.60-93.45 |
| | (h*ng/mL) | | | | | |
| | AUC_{∞} | 11 | 3009 | 2694 | 89.56 | 84.91-94.46 |
| | (h*ng/mL) | | | | | |

Treatment I: 25 mg milvexian (1 × 25 mg film-coated DC oral tablet; Ex. 17) BID for 5 days (administered in the morning after at least 10-hour overnight fasting and in the evening 12 hours after the morning dose)
Treatment J: 25 mg milvexian (1 × 25 mg SDP oral capsules; Compara. Ex. 2) BID for 5 days (administered in the morning after at least 10-hour overnight fasting and in the evening 12 hours after the morning dose)

In a second Phase 1 trial, Part 1 was an open-label, randomized, 3-way crossover study in healthy participants to evaluate the relative oral bioavailability, pharmacokinetics, and food effect of a single oral dose of 200 mg milvexian as 2×100 mg DC tablet Ex.18 compared to 200 mg capsule Compara. Ex. 1 under fasting conditions and to assess the effect of food on the bioavailability of milvexian after a single dose of 200 mg milvexian as 2×100 mg DC tablet Ex.18. Part 2 was an open-label, randomized, 2-way crossover Study in healthy participants to evaluate the PK and relative bioavailability of a single oral dose of 50 mg milvexian as 2×25 mg DC tablet Ex. 17 compared to 50 mg milvexian as 2×25 mg granule capsule Compara. Ex.2 in healthy participants under fasting conditions.

1. A solid pharmaceutical composition for oral administration comprising:
a. a spray-dried amorphous solid dispersion (SDP) consisting essentially of milvexian free form and a pH-dependent enterosoluble polymer;
b. a binder that is microcrystalline cellulose (MCC), silicified microcrystalline cellulose (SMCC), or a combination thereof;
c. a filler that is lactose monohydrate;
d. a disintegrant; and
e. a lubricant;
wherein milvexian free form is present in an amount ranging from about 10 wt. % to about 40 wt. % of the total weight of the solid pharmaceutical composition; and
wherein the binder and lactose monohydrate are present in a weight ratio (binder:lactose monohydrate) ranging from about 3:2 to about 3:1.

2. The solid pharmaceutical composition of claim 1, wherein the pH-dependent enterosoluble polymer is soluble in an aqueous medium at a pH of from about 5.5 to about 6.8.

3. The solid pharmaceutical composition of claim 1, wherein the solid pharmaceutical composition comprises from about 11.0 wt. % to about 21.0 wt. % of milvexian free form.

4. (canceled)

5. The solid pharmaceutical composition of claim 1, wherein the pH-dependent enterosoluble polymer is hydroxypropyl methyl cellulose-AS MG.

6. The solid pharmaceutical composition of claim 1, wherein the spray-dried amorphous solid dispersion has a median particle size distribution of D_{v,50}≤45 μm.

7. The solid pharmaceutical composition of claim 1, wherein the solid pharmaceutical composition comprises about 13.3 wt. % to about 53.3 wt. % of the spray-dried amorphous solid dispersion.

8. (canceled)

9. (canceled)

10. The solid pharmaceutical composition of claim 1, wherein the binder is about 21.0 wt. % to about 71.0 wt. % of silicified microcrystalline cellulose.

11.-14. (canceled)

15. The solid pharmaceutical composition of claim 1, wherein the solid pharmaceutical composition comprises about 25.0 wt. % to about 33.0 wt. % lactose monohydrate.

16. (canceled)

17. The solid pharmaceutical composition of claim 1, wherein the disintegrant is about 3.0 wt. % to about 7.0 wt. % of croscarmellose sodium.

18. (canceled)

19. (canceled)

20. The solid pharmaceutical composition of claim 1, wherein the lubricant is about 0.5 wt. % to about 2.0 wt. % of magnesium stearate.

21. (canceled)

22. The solid pharmaceutical composition of claim 1, wherein the composition comprises about 1.0 wt. % (wt/wt) of magnesium stearate.

23. The solid pharmaceutical composition of claim 1, consisting essentially of:
a) about 11.0 wt. % to about 21.0 wt. % of milvexian free form,
b) about 14.67 wt. % to about 28.0 wt. % of spray-dried amorphous solid dispersion (SDP),
c) about 38.0 wt. % to about 48.0 wt. % of Silicified Microcrystalline cellulose (SMCC 90),
d) about 25.0 wt. % to about 33.0 wt. % of lactose monohydrate,
e) about 3.0% wt. % to about 7.0 wt. % of croscarmellose sodium; and
f) about 0.5 wt. % to about 1.5 wt. % magnesium stearate.

24. The solid pharmaceutical composition of claim 1, consisting essentially of
a) about 16.67 wt. % of milvexian free form,
b) about 22.22 wt. % of spray-dried amorphous solid dispersion (SDP),
c) about 43.07 wt. % of silicified microcrystalline cellulose (SMCC 90),
d) about 28.71 wt. % of lactose monohydrate,
e) about 5.0% wt. % of croscarmellose sodium; and
f) about 1.0 wt. % of magnesium stearate.

25. The solid pharmaceutical composition of claim 1, wherein the solid pharmaceutical composition is a free flowing powder blend, wherein the free flowing powder blend has a tap density of about 0.56 g/mL; or a bulk density of about 0.47 g/mL; or a Flow function coefficient (ring shear) of 10.28; or an angle of repose of 50.17.

26.-32. (canceled)

33. A pharmaceutical tablet core formed by direct compression of the pharmaceutical composition of claim 1, and a film coating that coats the outside surface of the pharmaceutical tablet core.

34. (canceled)

35. The film-coated pharmaceutical tablet of claim 33, wherein the film coating comprises polyvinyl alcohol, titanium dioxide, polyethylene glycol-polyvinyl alcohol graft copolymer, and talc; or wherein the film coating comprises polyethylene glycol-polyvinyl alcohol graft copolymer.

36. (canceled)

37. The film-coated pharmaceutical tablet of claim 33, wherein the film coating comprises about 2.0% to about 4.0% of weight-gain by the uncoated tablet.

38. (canceled)

39. A pharmaceutical tablet comprising:
a. a core comprising:
i. a spray-dried amorphous solid dispersion (SDP) consisting essentially of milvexian free form and a pH-dependent enterosoluble polymer;
ii. a binder selected from microcrystalline cellulose (MCC), silicified microcrystalline cellulose (SMCC), or a combination thereof,
iii. lactose monohydrate;
iv. a disintegrant;
v. a lubricant; and
b. a film coating covering the core comprising polyvinyl alcohol, titanium dioxide, polyethylene glycol-polyvinyl alcohol graft copolymer, and talc; or a film coating comprising polyethylene glycol-polyvinyl alcohol graft copolymer.
wherein milvexian is present in an amount ranging from about 10 wt. % to about 40 wt. % of the total weight of the core; and
wherein the binder and lactose monohydrate are present in the core in a weight ratio (binder:lactose monohydrate) ranging from about 3:2 to about 3:1.

40. The pharmaceutical tablet of claim 39, wherein the tablet comprises about 25 mg of milvexian; or wherein the tablet comprises about 100 mg of milvexian.

41. (canceled)

42. The pharmaceutical tablet of claim 39, wherein the tablet has a friability of less than 0.5%; or wherein the tablet has a disintegration time of less than 2 minutes.

43-46. (canceled)
